SEQUENCE LISTING (132 sequences)

SEQ ID NO: 1 - DNA, 57 bp, synthetic construct
  source 1..57: mol_type = other DNA
acactctttccctacacgacgctcttccgatcttgacatgttcaatgtttgttctgc

SEQ ID NO: 2 - DNA, 53 bp, synthetic construct
  source 1..53: mol_type = other DNA
gtgactggagttcagacgtgtgctcttccgatctgtctgtccgttgctccatc

SEQ ID NO: 3 - DNA, 52 bp, synthetic construct
  source 1..52: mol_type = other DNA
acactctttccctacacgacgctcttccgatctcaaccctgtcacccacatc

SEQ ID NO: 4 - DNA, 55 bp, synthetic construct
  source 1..55: mol_type = other DNA
gtgactggagttcagacgtgtgctcttccgatcttgagcataacaagcagagtcc

SEQ ID NO: 5 - DNA, 54 bp, synthetic construct
  source 1..54: mol_type = other DNA
acactctttccctacacgacgctcttccgatctttgggattcaggaagtccaac

SEQ ID NO: 6 - DNA, 51 bp, synthetic construct
  source 1..51: mol_type = other DNA
gtgactggagttcagacgtgtgctcttccgatctaccagacagtggccaag

SEQ ID NO: 7 - DNA, 56 bp, synthetic construct
  source 1..56: mol_type = other DNA
acactctttccctacacgacgctcttccgatctccagacacaatcatacaggtgtg

SEQ ID NO: 8 - DNA, 56 bp, synthetic construct
  source 1..56: mol_type = other DNA
gtgactggagttcagacgtgtgctcttccgatctccagcctcctttggttagcaag

SEQ ID NO: 9 - DNA, 55 bp, synthetic construct
  source 1..55: mol_type = other DNA
acactctttccctacacgacgctcttccgatctatcaccagactgttgttgcttg

SEQ ID NO: 10 - DNA, 56 bp, synthetic construct
  source 1..56: mol_type = other DNA
gtgactggagttcagacgtgtgctcttccgatctctagtccagaggtggctagatg

SEQ ID NO: 11 - DNA, 53 bp, synthetic construct
  source 1..53: mol_type = other DNA
acactctttccctacacgacgctcttccgatcttaggaccctggctagcttga

SEQ ID NO: 12 - DNA, 54 bp, synthetic construct
  source 1..54: mol_type = other DNA
gtgactggagttcagacgtgtgctcttccgatctctctgatcacaggcactcca

SEQ ID NO: 13 - DNA, 61 bp, synthetic construct
  source 1..61: mol_type = other DNA
acactctttccctacacgacgctcttccgatcttacctgagatgttttgatataggcatcc

SEQ ID NO: 14 - DNA, 57 bp, synthetic construct
  source 1..57: mol_type = other DNA
gtgactggagttcagacgtgtgctcttccgatctgacccccatctctacaaaatgca

SEQ ID NO: 15 - DNA, 53 bp, synthetic construct
  source 1..53: mol_type = other DNA
acactctttccctacacgacgctcttccgatctacatgatgtctggcaccaga

SEQ ID NO: 16 - DNA, 54 bp, synthetic construct
  source 1..54: mol_type = other DNA
gtgactggagttcagacgtgtgctcttccgatctcttggccatcgcttcataga

SEQ ID NO: 17 - DNA, 52 bp, synthetic construct
  source 1..52: mol_type = other DNA
acactctttccctacacgacgctcttccgatctcagagcctgcaaagcatgg

SEQ ID NO: 18 - DNA, 56 bp, synthetic construct
  source 1..56: mol_type = other DNA
gtgactggagttcagacgtgtgctcttccgatctatgtttgcctgggtcagattca

SEQ ID NO: 19 - DNA, 56 bp, synthetic construct
  source 1..56: mol_type = other DNA
acactctttccctacacgacgctcttccgatctctggtttctctaccataagggcc

SEQ ID NO: 20 - DNA, 59 bp, synthetic construct
  source 1..59: mol_type = other DNA
gtgactggagttcagacgtgtgctcttccgatctcatcaagaactttcccaaatgtggg

SEQ ID NO: 21 - DNA, 52 bp, synthetic construct
  source 1..52: mol_type = other DNA
acactctttccctacacgacgctcttccgatcttgctggtgggcaggcaggt

SEQ ID NO: 22 - DNA, 53 bp, synthetic construct
  source 1..53: mol_type = other DNA
gtgactggagttcagacgtgtgctcttccgatctgagcaagccctgagccatt

SEQ ID NO: 23 - DNA, 52 bp, synthetic construct
  source 1..52: mol_type = other DNA
acactctttccctacacgacgctcttccgatctctgggatttgatgctaggg

SEQ ID NO: 24 - DNA, 54 bp, synthetic construct
  source 1..54: mol_type = other DNA
gtgactggagttcagacgtgtgctcttccgatctttataacctggcccatcagc

SEQ ID NO: 25 - DNA, 53 bp, synthetic construct
  source 1..53: mol_type = other DNA
acactctttccctacacgacgctcttccgatctcagtactgtttgcccagcct

SEQ ID NO: 26 - DNA, 56 bp, synthetic construct
  source 1..56: mol_type = other DNA
gtgactggagttcagacgtgtgctcttccgatctctcaccgtgctggaagacactg

SEQ ID NO: 27 - DNA, 63 bp, synthetic construct
  source 1..63: mol_type = other DNA
acactctttccctacacgacgctcttccgatcttttagggtgtattatctcatatactttcag

SEQ ID NO: 28 - DNA, 62 bp, synthetic construct
  source 1..62: mol_type = other DNA
gtgactggagttcagacgtgtgctcttccgatctatctcttctggaggactgaaatatttcc

SEQ ID NO: 29 - DNA, 53 bp, synthetic construct
  source 1..53: mol_type = other DNA
acactctttccctacacgacgctcttccgatctctgctgtgaagggacatcac

SEQ ID NO: 30 - DNA, 60 bp, synthetic construct
  source 1..60: mol_type = other DNA
gtgactggagttcagacgtgtgctcttccgatctctctttcaaggcttcatacatcttcc

SEQ ID NO: 31 - DNA, 56 bp, synthetic construct
  source 1..56: mol_type = other DNA
acactctttccctacacgacgctcttccgatctatggtgtgtttagcattacaggc

SEQ ID NO: 32 - DNA, 59 bp, synthetic construct
  source 1..59: mol_type = other DNA
gtgactggagttcagacgtgtgctcttccgatctcaagaattatgcttggtacttggct

SEQ ID NO: 33 - DNA, 61 bp, synthetic construct
  source 1..61: mol_type = other DNA
acactctttccctacacgacgctcttccgatctatgccaaggggaaacttagaagttgttc

SEQ ID NO: 34 - DNA, 61 bp, synthetic construct
  source 1..61: mol_type = other DNA
gtgactggagttcagacgtgtgctcttccgatctgcctctaagaatctgattgatgatata

SEQ ID NO: 35 - DNA, 64 bp, synthetic construct
  source 1..64: mol_type = other DNA
acactctttccctacacgacgctcttccgatctccaatcttagttgatgaagtaaaacgtattg

SEQ ID NO: 36 - DNA, 55 bp, synthetic construct
  source 1..55: mol_type = other DNA
gtgactggagttcagacgtgtgctcttccgatctttggctatggaacgagtggtc

SEQ ID NO: 37 - DNA, 58 bp, synthetic construct
  source 1..58: mol_type = other DNA
acactctttccctacacgacgctcttccgatcttgtcataggaaaagaatttcctccc

SEQ ID NO: 38 - DNA, 62 bp, synthetic construct
  source 1..62: mol_type = other DNA
gtgactggagttcagacgtgtgctcttccgatctgtgctgtatgagctataaacttctattc

SEQ ID NO: 39 - DNA, 54 bp, synthetic construct
  source 1..54: mol_type = other DNA
acactctttccctacacgacgctcttccgatctgatgggaagacgatcccactg

SEQ ID NO: 40 - DNA, 59 bp, synthetic construct
  source 1..59: mol_type = other DNA
gtgactggagttcagacgtgtgctcttccgatctgtaagacaccgtgtaagatgtaagc

SEQ ID NO: 41 - DNA, 60 bp, synthetic construct
  source 1..60: mol_type = other DNA
acactctttccctacacgacgctcttccgatctcctttgataggtttgatatgtgactaa

SEQ ID NO: 42 - DNA, 61 bp, synthetic construct
  source 1..61: mol_type = other DNA
gtgactggagttcagacgtgtgctcttccgatctccttttctggagtgacatatagaagtt

SEQ ID NO: 43 - DNA, 54 bp, synthetic construct
  source 1..54: mol_type = other DNA
acactctttccctacacgacgctcttccgatctgaaggactcattgtgccatgc

SEQ ID NO: 44 - DNA, 59 bp, synthetic construct
  source 1..59: mol_type = other DNA
gtgactggagttcagacgtgtgctcttccgatcttgaacataccttgattcctgggcaa

SEQ ID NO: 45 - DNA, 58 bp, synthetic construct
  source 1..58: mol_type = other DNA
acactctttccctacacgacgctcttccgatctctgtatttagcggaagaagtcccta

SEQ ID NO: 46 - DNA, 62 bp, synthetic construct
  source 1..62: mol_type = other DNA
gtgactggagttcagacgtgtgctcttccgatcttgtatcaggatttaaaatgagagcttcc

SEQ ID NO: 47 - DNA, 52 bp, synthetic construct
  source 1..52: mol_type = other DNA
acactctttccctacacgacgctcttccgatcttgactctgtctgcaggagg

SEQ ID NO: 48 - DNA, 55 bp, synthetic construct
  source 1..55: mol_type = other DNA
gtgactggagttcagacgtgtgctcttccgatctgtcaggggcacatagtagctt

SEQ ID NO: 49 - DNA, 56 bp, synthetic construct
  source 1..56: mol_type = other DNA
acactctttccctacacgacgctcttccgatctaggctacactcttcttgaaggtg

SEQ ID NO: 50 - DNA, 54 bp, synthetic construct
  source 1..54: mol_type = other DNA
gtgactggagttcagacgtgtgctcttccgatctctggtccagtctcacaggtt

SEQ ID NO: 51 - DNA, 60 bp, synthetic construct
  source 1..60: mol_type = other DNA
acactctttccctacacgacgctcttccgatctgtcttctacgtcttgtccttctgcaag

SEQ ID NO: 52 - DNA, 58 bp, synthetic construct
  source 1..58: mol_type = other DNA
gtgactggagttcagacgtgtgctcttccgatctcattgcttcaaatagtagtcctcc

SEQ ID NO: 53 - DNA, 53 bp, synthetic construct
  source 1..53: mol_type = other DNA
acactctttccctacacgacgctcttccgatcttccaaggaggagtgtgtctg

SEQ ID NO: 54 - DNA, 54 bp, synthetic construct
  source 1..54: mol_type = other DNA
gtgactggagttcagacgtgtgctcttccgatcttgcttcagcaccggcttcag

SEQ ID NO: 55 - DNA, 55 bp, synthetic construct
  source 1..55: mol_type = other DNA
acactctttccctacacgacgctcttccgatctcatagaggctattcccagcagc

SEQ ID NO: 56 - DNA, 54 bp, synthetic construct
  source 1..54: mol_type = other DNA
gtgactggagttcagacgtgtgctcttccgatctcatctggtggctgtgggtac

SEQ ID NO: 57 - DNA, 59 bp, synthetic construct
  source 1..59: mol_type = other DNA
acactctttccctacacgacgctcttccgatctgagcttctcataggaacatactagcg

SEQ ID NO: 58 - DNA, 62 bp, synthetic construct
  source 1..62: mol_type = other DNA
gtgactggagttcagacgtgtgctcttccgatcttagatatctccatatcaattccaactcc

SEQ ID NO: 59 - DNA, 67 bp, synthetic construct
  source 1..67: mol_type = other DNA
acactctttccctacacgacgctcttccgatctgttatcatcaaccttcagtagttctatgttttga

SEQ ID NO: 60 - DNA, 67 bp, synthetic construct
  source 1..67: mol_type = other DNA
gtgactggagttcagacgtgtgctcttccgatctttttacattgaaatagaaaggaactgtcagcta

SEQ ID NO: 61 - DNA, 53 bp, synthetic construct
  source 1..53: mol_type = other DNA
acactctttccctacacgacgctcttccgatctagccactgtgtccagcttct

SEQ ID NO: 62 - DNA, 54 bp, synthetic construct
  source 1..54: mol_type = other DNA
gtgactggagttcagacgtgtgctcttccgatctttggatccccttgtcttctg

SEQ ID NO: 63 - DNA, 54 bp, synthetic construct
  source 1..54: mol_type = other DNA
acactctttccctacacgacgctcttccgatctatgagattctcctgcgtgggt

SEQ ID NO: 64 - DNA, 59 bp, synthetic construct
  source 1..59: mol_type = other DNA
gtgactggagttcagacgtgtgctcttccgatctggtagtgcagaactgggatacacac

SEQ ID NO: 65 - DNA, 56 bp, synthetic construct
  source 1..56: mol_type = other DNA
acactctttccctacacgacgctcttccgatctaagttctgggattacaggtatgg

SEQ ID NO: 66 - DNA, 56 bp, synthetic construct
  source 1..56: mol_type = other DNA
gtgactggagttcagacgtgtgctcttccgatctatcacgagtgtttggcacaagg

SEQ ID NO: 67 - DNA, 55 bp, synthetic construct
  source 1..55: mol_type = other DNA
acactctttccctacacgacgctcttccgatctagttgtgacagccttggggaca

SEQ ID NO: 68 - DNA, 60 bp, synthetic construct
  source 1..60: mol_type = other DNA
gtgactggagttcagacgtgtgctcttccgatcttgcaattgctacttacatgagaaccg

SEQ ID NO: 69 - DNA, 58 bp, synthetic construct
  source 1..58: mol_type = other DNA
acactctttccctacacgacgctcttccgatctgctgttatatgctgtttgcaaactc

SEQ ID NO: 70 - DNA, 59 bp, synthetic construct
  source 1..59: mol_type = other DNA
gtgactggagttcagacgtgtgctcttccgatcttgttaatcaagttacagataccagc

SEQ ID NO: 71 - DNA, 59 bp, synthetic construct
  source 1..59: mol_type = other DNA
acactctttccctacacgacgctcttccgatctaagaaactgatcacatgtggaatgga

SEQ ID NO: 72 - DNA, 65 bp, synthetic construct
  source 1..65: mol_type = other DNA
gtgactggagttcagacgtgtgctcttccgatctgtgttagctaataatggagtcttgaaagaga

SEQ ID NO: 73 - DNA, 55 bp, synthetic construct
  source 1..55: mol_type = other DNA
acactctttccctacacgacgctcttccgatctgtctccttaaacctgtcttgta

SEQ ID NO: 74 - DNA, 54 bp, synthetic construct
  source 1..54: mol_type = other DNA
gtgactggagttcagacgtgtgctcttccgatctccagtgccagaagagccaag

SEQ ID NO: 75 - DNA, 59 bp, synthetic construct
  source 1..59: mol_type = other DNA
acactctttccctacacgacgctcttccgatctgggaaagaaaacatcaagcgtcccat

SEQ ID NO: 76 - DNA, 59 bp, synthetic construct
  source 1..59: mol_type = other DNA
gtgactggagttcagacgtgtgctcttccgatctgacagagaagactcttgggtttctg

SEQ ID NO: 77 - DNA, 55 bp, synthetic construct
  source 1..55: mol_type = other DNA
acactctttccctacacgacgctcttccgatctactgacctcccacattcccttt

SEQ ID NO: 78 - DNA, 58 bp, synthetic construct
  source 1..58: mol_type = other DNA
gtgactggagttcagacgtgtgctcttccgatcttcatgttcatacctcttatcttcc

SEQ ID NO: 79 - DNA, 56 bp, synthetic construct
  source 1..56: mol_type = other DNA
acactctttccctacacgacgctcttccgatcttagcttggactcagaataatcca

SEQ ID NO: 80 - DNA, 57 bp, synthetic construct
  source 1..57: mol_type = other DNA
gtgactggagttcagacgtgtgctcttccgatctcctaatctctttctttcagggca

SEQ ID NO: 81 - DNA, 24 bp, synthetic construct
  source 1..24: mol_type = other DNA
aaagttgtcacccatacatgccct

SEQ ID NO: 82 - DNA, 23 bp, synthetic construct
  source 1..23: mol_type = other DNA
ggaagctgaagcaagagaatcgc

SEQ ID NO: 83 - DNA, 24 bp, synthetic construct
  source 1..24: mol_type = other DNA
caaagcagacctcagacctcttac

SEQ ID NO: 84 - DNA, 20 bp, synthetic construct
  source 1..20: mol_type = other DNA
ctgggcttacgtcgctgtaa

SEQ ID NO: 85 - DNA, 53 bp, synthetic construct
  source 1..53: mol_type = other DNA
acactctttccctacacgacgctcttccgatctcatgcccagtttctattggt

SEQ ID NO: 86 - DNA, 54 bp, synthetic construct
  source 1..54: mol_type = other DNA
gtgactggagttcagacgtgtgctcttccgatctggtttgaagtccaactccta

SEQ ID NO: 87 - DNA, 56 bp, synthetic construct
  source 1..56: mol_type = other DNA
acactctttccctacacgacgctcttccgatctagaaggggaaagaaaacatcaag

SEQ ID NO: 88 - DNA, 59 bp, synthetic construct; identical to SEQ ID NO: 76
  source 1..59: mol_type = other DNA

SEQ ID NO: 89 - DNA, 52 bp, synthetic construct
  source 1..52: mol_type = other DNA
acactctttccctacacgacgctcttccgatctccaaggtttgaactagctc

SEQ ID NO: 90 - DNA, 54 bp, synthetic construct
  source 1..54: mol_type = other DNA
gtgactggagttcagacgtgtgctcttccgatctggattattctgagtccaagc

SEQ ID NO: 91 - DNA, 19 bp, synthetic construct
  source 1..19: mol_type = other DNA
cccctcgccaagtccaccc

SEQ ID NO: 92 - DNA, 19 bp, synthetic construct
  source 1..19: mol_type = other DNA
agaccaggaagggccggtg

SEQ ID NO: 93 - DNA, 21 bp, synthetic construct
  source 1..21: mol_type = other DNA
ggtttacccatgtggtgcctc

SEQ ID NO: 94 - DNA, 23 bp, synthetic construct
  source 1..23: mol_type = other DNA
cccgttggatcttctcatttccc

SEQ ID NO: 95 - DNA, 21 bp, synthetic construct
  source 1..21: mol_type = other DNA
aaagcactctagggtccagcg

SEQ ID NO: 96 - DNA, 25 bp, synthetic construct
  source 1..25: mol_type = other DNA
gcacaaatttctaggatgagtgtgg

SEQ ID NO: 97 - DNA, 21 bp, synthetic construct
  source 1..21: mol_type = other DNA
cgatctgggctctgtgttctc

SEQ ID NO: 98 - DNA, 21 bp, synthetic construct
  source 1..21: mol_type = other DNA
agcccacgttgtgttcatggc

SEQ ID NO: 99 - DNA, 22 bp, synthetic construct
  source 1..22: mol_type = other DNA
taccctttgcaagcacacgtac

SEQ ID NO: 100 - DNA, 21 bp, synthetic construct
  source 1..21: mol_type = other DNA
tcaatctccgacagctccgac

SEQ ID NO: 101 - DNA, 21 bp, synthetic construct
  source 1..21: mol_type = other DNA
gaccattcctcagcgtgggtg

SEQ ID NO: 102 - DNA, 21 bp, synthetic construct
  source 1..21: mol_type = other DNA
caagtgggctgagcccttgag

SEQ ID NO: 103 - DNA, 24 bp, synthetic construct
  source 1..24: mol_type = other DNA
tttaaatgggcaaaacaggccagg

SEQ ID NO: 104 - DNA, 25 bp, synthetic construct
  source 1..25: mol_type = other DNA
ataacctttatctgccacatgtagc

SEQ ID NO: 105 - DNA, 22 bp, synthetic construct
  source 1..22: mol_type = other DNA
gtcgtcactggcagcgtagatc

SEQ ID NO: 106 - DNA, 23 bp, synthetic construct
  source 1..23: mol_type = other DNA
gattccaggttgtagacggactg

SEQ ID NO: 107 - DNA, 56 bp, synthetic construct
  source 1..56: mol_type = other DNA
acactctttccctacacgacgctcttccgatctcagtcattcctcattctgtcctc

SEQ ID NO: 108 - DNA, 55 bp, synthetic construct
  source 1..55: mol_type = other DNA
gtgactggagttcagacgtgtgctcttccgatcttaccaccagcttactggaagg

SEQ ID NO: 109 - DNA, 54 bp, synthetic construct
  source 1..54: mol_type = other DNA
acactctttccctacacgacgctcttccgatctttgcctttgtccttaccctag

SEQ ID NO: 110 - DNA, 57 bp, synthetic construct
  source 1..57: mol_type = other DNA
gtgactggagttcagacgtgtgctcttccgatctacatgggctgtttgtaaaacgtg

SEQ ID NO: 111 - DNA, 57 bp, synthetic construct
  source 1..57: mol_type = other DNA
acactctttccctacacgacgctcttccgatctgacttcttagatgagggtttcatg

SEQ ID NO: 112 - DNA, 53 bp, synthetic construct
  source 1..53: mol_type = other DNA
gtgactggagttcagacgtgtgctcttccgatctgctgtcttcagcccacttc

SEQ ID NO: 113 - DNA, 57 bp, synthetic construct
  source 1..57: mol_type = other DNA
acactctttccctacacgacgctcttccgatctcaggatcacactctcagcttctcc

SEQ ID NO: 114 - DNA, 56 bp, synthetic construct
  source 1..56: mol_type = other DNA
gtgactggagttcagacgtgtgctcttccgatctcagcgtgtgagctgactctgtc

SEQ ID NO: 115 - DNA, 56 bp, synthetic construct
  source 1..56: mol_type = other DNA
acactctttccctacacgacgctcttccgatctctttgaataaagtctgagtgggc

SEQ ID NO: 116 - DNA, 55 bp, synthetic construct
  source 1..55: mol_type = other DNA
gtgactggagttcagacgtgtgctcttccgatctaaagcactctagggtccagcg

SEQ ID NO: 117 - DNA, 53 bp, synthetic construct
  source 1..53: mol_type = other DNA
acactctttccctacacgacgctcttccgatctcagaccaaggacctctctgc

SEQ ID NO: 118 - DNA, 54 bp, synthetic construct
  source 1..54: mol_type = other DNA
gtgactggagttcagacgtgtgctcttccgatctgaagtgtgagtagggccagc

SEQ ID NO: 119 - DNA, 54 bp, synthetic construct
  source 1..54: mol_type = other DNA
acactctttccctacacgacgctcttccgatctcgatctgggctctgtgttctc

SEQ ID NO: 120 - DNA, 54 bp, synthetic construct
  source 1..54: mol_type = other DNA
gtgactggagttcagacgtgtgctcttccgatctctccagaacatttggcgatt

SEQ ID NO: 121 - DNA, 53 bp, synthetic construct
  source 1..53: mol_type = other DNA
acactctttccctacacgacgctcttccgatcttggcacctaccctggaaaca

SEQ ID NO: 122 - DNA, 54 bp, synthetic construct
  source 1..54: mol_type = other DNA
gtgactggagttcagacgtgtgctcttccgatctagggaaggcaagtccagagg

SEQ ID NO: 123 - DNA, 58 bp, synthetic construct
  source 1..58: mol_type = other DNA
acactctttccctacacgacgctcttccgatctgcacaaatttctaggatgagtgtgg

SEQ ID NO: 124 - DNA, 55 bp, synthetic construct
  source 1..55: mol_type = other DNA
gtgactggagttcagacgtgtgctcttccgatctcatgtactccggatgttgggc

SEQ ID NO: 125 - DNA, 55 bp, synthetic construct
  source 1..55: mol_type = other DNA
acactctttccctacacgacgctcttccgatcttaccctttgcaagcacacgtac

SEQ ID NO: 126 - DNA, 54 bp, synthetic construct
  source 1..54: mol_type = other DNA
gtgactggagttcagacgtgtgctcttccgatctgaaggtgaggtcatgcaggc

SEQ ID NO: 127 - DNA, 57 bp, synthetic construct
  source 1..57: mol_type = other DNA
acactctttccctacacgacgctcttccgatcttttaaatgggcaaaacaggccagg

SEQ ID NO: 128 - DNA, 59 bp, synthetic construct
  source 1..59: mol_type = other DNA
gtgactggagttcagacgtgtgctcttccgatctataacctttatctgccacatgtagc

SEQ ID NO: 129 - DNA, 53 bp, synthetic construct
  source 1..53: mol_type = other DNA
acactctttccctacacgacgctcttccgatctggcgagatggcgccttcctc

SEQ ID NO: 130 - DNA, 53 bp, synthetic construct
  source 1..53: mol_type = other DNA
gtgactggagttcagacgtgtgctcttccgatctagaccaggaagggccggtg

SEQ ID NO: 131 - DNA, 57 bp, synthetic construct
  source 1..57: mol_type = other DNA
acactctttccctacacgacgctcttccgatctagcatgaattgtttaagggtaagc

SEQ ID NO: 132 - DNA, 57 bp, synthetic construct
  source 1..57: mol_type = other DNA
gtgactggagttcagacgtgtgctcttccgatctaactcaactcagcagcaggctgt

(19) United States
(12) Patent Application Publication
(10) Pub. No.: US 2025/0043350 A1
(43) Pub. Date: Feb. 6, 2025

(54) METHODS FOR DETECTING INHERITED MUTATIONS USING MULTIPLEX GENE SPECIFIC PCR

(71) Applicant: QUEST DIAGNOSTICS INVESTMENTS LLC, Secaucus, NJ (US)

(72) Inventor: Sun Hee Rosenthal, Secaucus, NJ (US)

(73) Assignee: QUEST DIAGNOSTICS INVESTMENTS LLC, Secaucus, NJ (US)

(21) Appl. No.: 18/717,114

(22) PCT Filed: Dec. 6, 2022
(86) PCT No.: PCT/US2022/052044

§ 371 Date: Jun. 6, 2024

Related U.S. Application Data
(60) Provisional application No. 63/286,906, filed on Dec. 7, 2021.

(51) Int. Cl. C12Q 1/6883

(52) U.S. Cl. CPC C12Q 1/6883; C12Q 2600/156; C12Q 2600/16

(57) ABSTRACT

The present disclosure provides methods for detecting inherited mutations (e.g., Ashkenazi Jewish carrier mutations, beta thalassemia mutations or an alpha thalassemia mutations) using multiplex gene-specific PCR. Kits for use in practicing the methods are also provided.

### CROSS REFERENCE TO RELATED APPLICATION

This application claims the benefit under 35 U.S.C. § 119 (e) of U.S. Provisional Application No. 63/286,906, filed Dec. 7, 2021, the entire contents of which are incorporated herein by reference.

### TECHNICAL FIELD

The present disclosure provides methods for detecting inherited mutations (e.g., Ashkenazi Jewish carrier mutations, beta thalassemia mutations or an alpha thalassemia mutations) using multiplex gene-specific PCR. Kits for use in practicing the methods are also provided.

### BACKGROUND

The following description of the background of the present disclosure is provided simply to aid the reader in understanding the disclosure and is not admitted to describe or constitute prior art to the present disclosure.

Thalassemia is an inherited autosomal recessive disease resulting from mutations in the α- and β-globin gene clusters on chromosome 16 and chromosome 11, respectively. It is characterized by the absence or reduced synthesis of globin chains of hemoglobin and includes two main types, α- and β-thalassemia. It is reported thalassemia is one of the top five kinds of major birth defects. Thalassemia major has imposed an enormous burden on society and has serious impact on the quality of life of the impacted population. High prevalence of thalassemia is observed in southern China, Southeast Asia, India, the Middle East, Africa and the Mediterranean region.

The Ashkenazi Jewish population that lived mainly in central and Eastern Europe maintained a genetic isolation, separated from its neighbors by religious and cultural practices as well as consanguinity. The main evidence for this isolation of the Ashkenazi Jewish population is the existence of genetic characteristics, including a high prevalence of autosomal-recessive diseases and a relatively high frequency of alleles that confer a risk to common disorders such as breast and ovarian cancer, or variants associated with inflammatory bowel disease or for Parkinson disease.

There is a substantial need for more robust and sensitive methods that can rapidly detect whether a subject is a carrier for inherited genetic diseases (including diseases that are frequently found in the Ashkenazi Jewish population, beta thalassemia and alpha thalassemia) in a single biological sample.

### SUMMARY

The present disclosure provides methods for simultaneous identification of inherited genetic variants (such as Ashkenazi Jewish panel variations, beta thalassemia variations and alpha thalassemia variations). The disclosure also provides methods for identifying Ashkenazi Jewish panel variations, beta thalassemia variations and alpha thalassemia variations in a biological sample from a subject. Finally, the disclosure provides kits for practicing the methods described herein.

In some embodiments, the method comprises: extracting DNA from a biological sample obtained from the subject; generating a first plurality of amplicons by contacting the biological sample with a first plurality of primer pairs, wherein at least one amplicon corresponds to each of a plurality of genes, said plurality of genes comprising HEXA, SMPD1, MCOLN1, GBA, FANCC, IKBKAP, ASPA, BLM, BCKDHB, G6PC, ABCC8, DLD, NEB, PCDH15, CLRN1, TMEM216, and FKTN; generating a second plurality of amplicons by contacting the biological sample with a second plurality of primer pairs, wherein at least one amplicon corresponds to each of GBA and HBB, wherein the second plurality of primer pairs comprises SEQ ID NOs: 81 and 82, and SEQ ID NOs: 83 and 84; and generating a third plurality of amplicons by contacting the biological sample with a third plurality of primer pairs, a hotstart Taq DNA polymerase, and a Taq Extender PCR additive, wherein at least one amplicon corresponds to each of HBA1 and HBA2; incorporating a barcode sequence on to the ends of the first, second and third plurality of amplicons via a polymerase chain reaction; and detecting one or more variants in at least one of the first, second and third plurality of amplicons using high throughput massive parallel sequencing.

In some embodiments, the Ashkenazi Jewish panel variations comprise gene variations (e.g., single nucleotide variations (SNVs), deletions, insertions, or inversions) that are commonly observed in the Ashkenazi Jewish population, but that may also occur outside the Ashkenazi Jewish population as well, albeit in lower frequencies. In some embodiments, the Ashkenazi Jewish panel variations comprise genetic variations that can cause Tay-Sachs disease, Niemann Pick disease, Mucolipidosis Type IV, Gaucher disease, Fanconi Anemia, Familial Dysautonomia, Canavan disease, Bloom syndorme, Maple serum urinary disease, Glycogen storage disease I, Familial Hyperinsulinism, Dihydrolipoamide Dehydrogenase Deficiency (DLD), Lipoamide Dehydrogenase Deficiency (E3), Nemaline Myopathy, Usher Syndrome Type IF, Usher Syndrome, Type IIIA, Joubert Syndrome 2, or Walker-Warburg Syndrome. In some embodiments, the Ashkenazi Jewish panel variations comprise genetic variations listed in Table 1. In some embodiments, the beta thalassemia variations comprise genetic variations listed in Table 2. In some embodiments, the beta thalassemia variations comprise genetic variations listed in Table 3.

In some embodiments, the first plurality of primer pairs comprises at least one gap primer pair designed to amplify target regions only when a deletion is present. In some embodiments, the GBA primers of the second plurality of primer pairs specifically amplify the GBA gene and not amplify any GBA pseudogenes. In some embodiments, the third plurality of primer pairs comprises at least one gap primer pair designed to amplify target regions only when a deletion is present.

In some embodiments, the HEXA gene variations cause Tay-Sachs disease; and the SMPD1 gene variations cause Niemann Pick disease; and the MCOLN1 gene variations cause Mucolipidosis Type IV; and the GBA gene variations cause Gaucher disease; and the PANCC gene variations cause Fanconi Anemia; and the IKBKAP gene variations cause Familial Dysautonomia; and the ASPA gene variations cause Canavan disease; and the BLM gene variations cause Bloom syndorme; and the BCKDHB gene variations cause Maple serum urinary disease; and the G6PC gene variations cause Glycogen storage disease I; and the ABCC8 gene variations cause Familial Hyperinsulinism; and the DLD gene variations cause Dihydrolipoamide Dehydrogenase Deficiency (DLD) or Lipoamide Dehydrogenase Deficiency (E3); and the NEB gene variations cause Nemaline Myopathy; and the PCDH15 gene variations cause Usher Syndrome, Type IF; and the CLRN1 gene variations cause Usher Syndrome, Type IIIA; and the TMEM216 gene variations cause Joubert Syndrome 2; and the FKTN gene variations cause Walker-Warburg Syndrome.

In some embodiments, the Ashkenazi Jewish panel gene variations comprise one or more gene variations recited in Table 1. In some embodiments, the HEXA gene variations comprise one or more of R178H (B1 variant), R247W, R249W, G269S, IVS9+1G>A, 1278_TATC, IVS12+1G>C, or 7.6-kb Del, Ex1; and the SMPD1 gene variations comprise one or more of L302P, fsP330, R496L (R498L), or deltaR608; and the MCOLN1 gene variations comprise one or more of IVS3−2A>G, or 6.4 kb_del; and the GBA gene variations comprise one or more of IVS2+1G>A, 84G>GG, N370S, del_55 bp, V394L, D409H, L444P, or R496H; and the PANCC gene variations comprise one or more of IVS4+4A>T, or 322delG; and the IKBKAP gene variations comprise one or more of R696P, or IVS20+6T>C; and the ASPA gene variations comprise one or more of IVS2−2A>G, Y231X, E285A, or A305E; and the BLM gene variations comprise one or more of 2281del6/ins7; and the BCKDHB gene variations comprise one or more of R183P, G278S, or E372X; and the G6PC gene variations comprise one or more of R83C or Q347X; and the ABCC8 gene variations comprise IVS32−9G>A or F1387del; and the DLD gene variations comprise one or more of Y35* or G229C; and the NEB gene variations comprise R2478_D2512del35; and the PCDH15 gene variations comprise one or more of R245; and the CLRN1 gene variations comprise N48K; and the TMEM216 gene variations comprise R73L; and the FKTN gene variations comprise F390fs.

In some embodiments, the beta thalassemia gene variations in the HBB gene comprise one or more gene variations recited in Table 2. In some embodiments, the gene variations comprise c.*111A>G, c.*110T>C, c.*96T>C, Hb D-Los Angeles (c.364G>C), Hb O-Arab (c.364G>A), c.321_322insG, c.316−2A>C, c.316−2A>G, c.316−3C>A, c.316−106C>G, c.316−125A>G, c.316−146T>G, c.316−197C>T, c.315+1G>A, c.287_288insA, c.251 delG, c.230delC, c.216_217insA, c.203_204delTG, c.143_144insA, c.146_147insATCT, c.135delC, c.130G>T, c.126_129delCTTT, c.124_127delTTCT, c.118C>T, c.114G>A, c.112delT, c.93−1G>C, c.93−1G>A, c.93−21G>A, c.92+6T>C, c.92+5G>A, c.92+5G>C, c.92+5G>T, c.92+2T>A, c.92+2T>C, c.92+1G>A, c.92+1G>T, Hb Monroe (c.92G>C), c.92G>A, c.84_85insC, c.79G>T, HB E (c.79G>A), c.75T>A, c.59A>G, c.52A>T (LYS17*), c.51delC, c.48G>A, c.47G>A (Trp15), c.46delT, c.36delT, c.33C>A, c.27_28insG, c.25_26delAA, c.20delA, HB S (c.20A>T), HB C (c.19G>A), c.17_18delCT, c.2T>C, c.2T>G, c.1A>G, c.−78A>C, c.−78A>G, c.−79A>G, c.−80T>A, c.−81A>G, c.−136C>G, c.−137C>A, c.−137C>G, c.−137C>T, c.−138C>T, c.−138C>A, c.−140C>T, or c.−151C>T.

In some embodiments, the alpha thalassemia variations in the HBA1 and HBA2 genes comprise one or more variations recited in Table 3. In some embodiments, the alpha thalassemia variations in the HBA1 and HBA2 genes comprise large deletions selected from variant 3.7, variant 4.2, variant SEA, variant THAI, variant 20.5, variant MED or variant FIL; or a single nucleotide variant in the HBA2 gene HBA2 c.427T>C.

In some embodiments, the first plurality of primer pairs comprises at least two, at least four, at least five, at least eight or at least ten primer pairs having at least 85%, at least 88%, at least 90%, at least 93%, at least 95%, at least 99%, or more identity to at least two, at least four, at least five, at least eight or at least ten, at least 15, at least 20, at least 25, at least 30, at least 35, or all primer pairs selected from Table 4. In some embodiments, the first plurality of primer pairs comprises all the primer pairs shown in Table 4.

In some embodiments, the second plurality of primer pairs comprises at least two, at least four, or at least five primer pairs having at least 85%, at least 88%, at least 90%, at least 93%, at least 95%, at least 99%, or more identity to at least two, at least four, or at least five primer pairs selected from Table 5.

In some embodiments, the third plurality of primer pairs comprises at least two, at least four, at least five, at least eight or at least ten primer pairs having at least 85% identity to at least two, at least three or at least four primer pairs selected from Table 6. In some embodiments, the first plurality of primer pairs comprises all the primer pairs shown in Table 6.

In some embodiments, the next generation sequencing comprises sequencing by synthesis (e.g., pyrosequencing). In some embodiments, the next generation sequencing comprises sequencing by ligation.

In some embodiments, the subject is suspected of being a carrier for at least one disease selected from Tay-Sachs disease, Niemann Pick disease, Mucolipidosis Type IV, Gaucher disease, Fanconi Anemia, Familial Dysautonomia, Canavan disease, Bloom syndorme, Maple serum urinary disease, Glycogen storage disease I, Familial Hyperinsulinism, Dihydrolipoamide Dehydrogenase Deficiency (DLD), Lipoamide Dehydrogenase Deficiency (E3), Nemaline Myopathy, Usher Syndrome Type IF, Usher Syndrome, Type IIIA, Joubert Syndrome 2, Walker-Warburg Syndrome, beta thalassemia or alpha thalassemia.

In some embodiments, the subject is of Ashkenazi Jewish descent or has a family history of, beta thalassemia or alpha thalassemia.

In some embodiments, the biological sample is selected from whole blood, serum, plasma, amniotic fluid or chorionic villi.

In some embodiments, the genetic disorder is an autosomal or X-linked recessive disorder.

In another aspect, the present disclosure provides kits comprising oligonucleotides which may be primers or probes for performing amplifications as described herein.

The present disclosure also provides kits for detecting the presence of Ashkenazi Jewish panel variations, beta thalassemia variations or alpha thalassemia variations in a biological sample from a subject.

Kits of the present technology comprise at least (i) a first plurality of primer pairs directed to amplifying regions of each of a plurality of genes, said plurality of genes comprising HEXA, SMPD1, MCOLN1, GBA, FANCC, IKBKAP, ASPA, BLM, BCKDHB, G6PC, ABCC8, DLD, NEB, PCDH15, CLRN1, TMEM216, and FKTN; (ii) a second plurality of primer pairs directed to amplifying regions of GBA and HBB genes, wherein the second plurality of primer pairs comprises SEQ ID NOs: 81 and 82, and SEQ ID NOs: 83 and 84; and (iii) a third plurality of primer pairs directed to amplifying regions of alpha thalassemia gene variations in each of HBA1 and HBA2 genes, and instructions for use.

In some embodiments, the first plurality of primer pairs comprises at least one gap primer pair designed to amplify target regions only when a deletion is present.

In some embodiments, the GBA primers of the second plurality of primer pairs specifically amplify the GBA gene and not amplify any GBA pseudogenes.

In some embodiments, the third plurality of primer pairs comprises at least one gap primer pair designed to amplify target regions only when a deletion is present.

In some embodiments, the HEXA gene variations cause Tay-Sachs disease; and the SMPD1 gene variations cause Niemann Pick disease; and the MCOLN1 gene variations cause Mucolipidosis Type IV; and the GBA gene variations cause Gaucher disease; and the PANCC gene variations cause Fanconi Anemia; and the IKBKAP gene variations cause Familial Dysautonomia; and the ASPA gene variations cause Canavan disease; and the BLM gene variations cause Bloom syndorme; and the BCKDHB gene variations cause Maple serum urinary disease; and the G6PC gene variations cause Glycogen storage disease I; and the ABCC8 gene variations cause Familial Hyperinsulinism; and the DLD gene variations cause Dihydrolipoamide Dehydrogenase Deficiency (DLD) or Lipoamide Dehydrogenase Deficiency (E3); and the NEB gene variations cause Nemaline Myopathy; and the PCDH15 gene variations cause Usher Syndrome, Type IF; and the CLRN1 gene variations cause Usher Syndrome, Type IIIA; and the TMEM216 gene variations cause Joubert Syndrome 2; and the FKTN gene variations cause Walker-Warburg Syndrome.

In some embodiments, the Ashkenazi Jewish panel gene variations comprise one or more gene variations recited in Table 1. In some embodiments, the HEXA gene variations comprise one or more of R178H (B1 variant), R247W, R249W, G269S, IVS9+1G>A, 1278_TATC, IVS12+1G>C, or 7.6-kb Del, Ex1; and the SMPD1 gene variations comprise one or more of L302P, fsP330, R496L (R498L), or deltaR608; and the MCOLN1 gene variations comprise one or more of IVS3−2A>G, or 6.4 kb_del; and the GBA gene variations comprise one or more of IVS2+1G>A, 84G>GG, N370S, del_55 bp, V394L, D409H, L444P, or R496H; and the PANCC gene variations comprise one or more of IVS4+4A>T, or 322delG; and the IKBKAP gene variations co comprise one or more of mprise R696P, or IVS20+6T>C; and the ASPA gene variations comprise one or more of IVS2−2A>G, Y231X, E285A, or A305E; and the BLM gene variations comprise one or more of 2281 del6/ins7; and the BCKDHB gene variations comprise one or more of R183P, G278S, or E372X; and the G6PC gene variations comprise one or more of R83C or Q347X; and the ABCC8 gene variations comprise IVS32−9G>A or F1387del; and the DLD gene variations comprise one or more of Y35* or G229C; and the NEB gene variations comprise R2478_D2512del35; and the PCDH15 gene variations comprise one or more of R245; and the CLRN1 gene variations comprise N48K; and the TMEM216 gene variations comprise R73L; and the FKTN gene variations comprise F390fs.

In some embodiments, the beta thalassemia gene variations in the HBB gene comprise one or more gene variations recited in Table 2. In some embodiments, the gene variations comprise c.*111A>G, c.*110T>C, c.*96T>C, Hb D-Los Angeles (c.364G>C), Hb O-Arab (c.364G>A), c.321_322insG, c.316−2A>C, c.316−2A>G, c.316−3C>A, c.316−106C>G, c.316−125A>G, c.316−146T>G, c.316−197C>T, c.315+1G>A, c.287_288insA, c.251 delG, c.230delC, c.216_217insA, c.203_204delTG, c.143_144insA, c.146_147insATCT, c.135delC, c.130G>T, c.126_129delCTTT, c.124_127delTTCT, c.118C>T, c.114G>A, c.112delT, c.93−1G>C, c.93−1G>A, c.93−21G>A, c.92+6T>C, c.92+5G>A, c.92+5G>C, c.92+5G>T, c.92+2T>A, c.92+2T>C, c.92+1G>A, c.92+1G>T, Hb Monroe (c.92G>C), c.92G>A, c.84_85insC, c.79G>T, HB E (c.79G>A), c.75T>A, c.59A>G, c.52A>T (LYS17*), c.51delC, c.48G>A, c.47G>A (Trp15), c.46delT, c.36delT, c.33C>A, c.27_28insG, c.25_26delAA, c.20delA, HB S (c.20A>T), HB C (c.19G>A), c.17_18delCT, c.2T>C, c.2T>G, c.1A>G, c.−78A>C, c.−78A>G, c.−79A>G, c.−80T>A, c.−81A>G, c.−136C>G, c.−137C>A, c.−137C>G, c.−137C>T, c.−138C>T, c.−138C>A, c.−140C>T, or c.−151C>T.

In some embodiments, the alpha thalassemia variations in the HBA1 and HBA2 genes comprise one or more gene variations recited in Table 3. In some embodiments, the alpha thalassemia variations in the HBA1 and HBA2 genes comprise large deletions selected from variant 3.7, variant 4.2, variant SEA, variant THAI, variant 20.5, variant MED or variant FIL; or a single nucleotide variant in the HBA2 gene HBA2 c.427T>C.

In some embodiments, the first plurality of primer pairs comprises at least two, at least four, at least five, at least eight or at least ten primer pairs having at least 85%, at least 88%, at least 90%, at least 93%, at least 95%, at least 99%, or more identity to at least two, at least four, at least five, at least eight, at least 10, at least 15, at least 20, at least 25, at least 30, at least 35 or all primer pairs selected from Table 4. In some embodiments, the first plurality of primer pairs comprises all the primer pairs shown in Table 4.

In some embodiments, the second plurality of primer pairs comprises at least two, at least four, or at least five primer pairs having at least 85%, at least 88%, at least 90%, at least 93%, at least 95%, at least 99%, or more identity to at least two, at least four, or at least five primer pairs selected from Table 5. In some embodiments, the first plurality of primer pairs comprises all the primer pairs shown in Table 5.

In some embodiments, the third plurality of primer pairs comprises at least two, at least four, at least five, at least eight or at least ten primer pairs having at least 85% identity to at least two, at least four, at least five, at least eight or at least ten primer pairs selected from Table 6. In some embodiments, the first plurality of primer pairs comprises all the primer pairs shown in Table 6.

### BRIEF DESCRIPTION OF THE DRAWINGS

FIG. 1. Sequencing library preparation workflow.

FIG. 2. Sequencing data analysis workflow.

### DETAILED DESCRIPTION

The present disclosure provides a method (an “expanded carrier screen”) which offers hotspot molecular detection of variants for multiple autosomal and X-linked recessive disorders at the same time and allows testing of individuals regardless of ancestry or geographic origin. The instant carrier screening easily and quickly identifies couples who have an increased risk of having an affected child in order to facilitate informed reproductive decision making.

The carrier screening panel disclosed herein includes 20 genes associated with 19 diseases including: Alpha Thalassemia, Beta Thalassemia (including Sickle Cell anemia), Bloom Syndrome, Canavan Disease, Dihydrolipoamide Dehydrogenase Deficiency, Familial Dysautonomia, Familial Hyperinsulinism, Fanconi Anemia Type C, Gaucher Disease, Glycogen Storage disease Type IA, Joubert Syndrome 2, Maple Syrup Urine Disease, Mucolipidosis IV, Nemaline Myopathy, Niemann-Pick Disease Type A, Tay-Sachs Disease, Usher Syndrome Type IF, Usher Syndrome Type III, and Walker-Warburg Syndrome.

The instant carrier screening panel comprises 24 genes, by inclusion of 3 additional diseases, Cystic Fibrosis (CFTR), Fragile X Syndrome (FMR1), and Spinal Muscular Atrophy (SMN1 and SMN2) in addition to the aforementioned carrier screening panel of 19 disorders (20 genes).

The disclosed carrier screen offers hotspot molecular detection of variants for multiple autosomal and X-linked recessive disorders at the same time and allows testing of individuals regardless of ancestry or geographic origin. Carrier screening aims to identify couples who have an increased risk of having an affected child in order to facilitate informed reproductive decision making.

### Definitions

Unless otherwise defined, all technical and scientific terms used herein have the same meaning as commonly understood by one of ordinary skill in the art to which the present technology belongs.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” in reference to a number is generally taken to include numbers that fall within a range of 1%-10% in either direction (greater than or less than) of the number unless otherwise stated or otherwise evident from the context.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the terms “amplify” or “amplification” with respect to nucleic acid sequences, refer to methods that increase the representation of a population of nucleic acid sequences in a sample. Copies of a particular target nucleic acid sequence generated in vitro in an amplification reaction are called “amplicons” or “amplification products.” Amplification may be exponential or linear. A target nucleic acid may be DNA (such as, for example, genomic DNA and cDNA) or RNA. While the exemplary methods described hereinafter relate to amplification using polymerase chain reaction (PCR), numerous other methods such as isothermal methods, rolling circle methods, etc., are well known to the skilled artisan. The skilled artisan will understand that these other methods may be used either in place of, or together with, PCR methods. See, e.g., Saiki, “Amplification of Genomic DNA” in PCR PROTOCOLS, Innis et al., Eds., Academic Press, San Diego, CA 1990, pp 13-20; Wharam, et al., Nucleic Acids Res. 29 (11): E54-E54 (2001).

An “amplification mixture” as used herein is a mixture of reagents that are used in a nucleic acid amplification reaction, but does not contain primers or sample. An amplification mixture comprises a buffer, dNTPs, and a DNA polymerase. An amplification mixture may further comprise at least one of MgCl₂, KCl, nonionic and ionic detergents (including cationic detergents).

An “amplification master mix” comprises an amplification mixture and primers for amplifying one or more target nucleic acids, but does not contain the sample to be amplified.

As used herein, the phrase, “selectively amplifying” refers to an amplification reaction (e.g., a PCR reaction) in which only chosen sequences are amplified. The phrase “gene-specific amplification” refers to an amplification reaction (e.g., a PCR reaction) in which only chosen gene or part of a gene (e.g., where a known/preselected variant occurs) is amplified.

The terms “complement,” “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “substantially complementary” as used herein means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

As used herein, the term “detecting” refers to determining the presence of a target nucleic acid in the sample. Detection does not require the method to provide 100% sensitivity and/or 100% specificity.

As used herein, the term “direct amplification” refers to a nucleic acid amplification reaction in which the target nucleic acid is amplified from the sample without prior purification, extraction, or concentration.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by Illumina, Life Technologies, and Roche etc. (e.g., Illumina's reversible terminator method, Roche's pyrosequencing method (454), Life Technologies' sequencing by ligation (the SOLiD platform), or Pacific Biosciences' fluorescent base-cleavage method). Next-generation sequencing methods may also include nanopore sequencing methods or electronic-detection based methods such as Ion Torrent technology commercialized by Life Technologies. Examples of such methods are described in the following references: Margulies et al (Nature 2005 437:376-80); Ronaghi et al (Analytical Biochemistry 1996 242:84-9); Shendure (Science 2005 309:1728); Imelfort et al (Brief Bioinform. 2009 10:609-18); Fox et al (Methods Mol Biol. 2009; 553:79-108); Appleby et al (Methods Mol Biol. 2009; 513:19-39) English (PLOS One. 2012 7: e47768) and Morozova (Genomics. 2008 92:255-64), which are incorporated by reference for the general descriptions of the methods and the particular steps of the methods, including all starting products, reagents, and final products for each of the steps. Next generation sequencing may result in at least 10,000, at least 50,000, at least 100,000, at least 500,000, at least 1M at least 10M at least 100M or at least 1B sequence reads. In many cases, the reads are paired-end reads.

In some embodiments, the next-generation sequencing of the instant disclosure comprises the following next generation sequencing systems:

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX™ sequencing system (Roche, Germany), employs a light-based detection methodology in a large-scale parallel pyrosequencing system.

Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added poly A tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

The term “barcode sequence” or “molecular barcode,” as used herein, refers to a unique sequence of nucleotides used to (a) identify and/or track the source of a polynucleotide in a reaction and/or (b) count how many times an initial molecule is sequenced (e.g., in cases where substantially every molecule in a sample is tagged with a different sequence, and then the sample is amplified). A barcode sequence may be at the 5′-end, the 3′-end or in the middle of an oligonucleotide, or both the 5′ end and the 3′ end. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97:1665-1670 (2000); Shoemaker et al, Nature Genetics, 14:450-456 (1996); Morris et al, European patent publication 0799897A1; Wallace, U.S. Pat. No. 5,981,179; and the like. In some embodiments, a barcode sequence may have a length in range of from 4 to 36 nucleotides, or from 6 to 30 nucleotides, or from 8 to 20 nucleotides.

As used herein, the term “extraction” refers to any action taken to remove nucleic acids from other (non-nucleic acid) material present in the sample. The term extraction includes mechanical or chemical lysis, addition of detergent or protease, or precipitation and removal of non-nucleic acids such as proteins.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

A “hot start”, in the context of a nucleic acid amplification reaction, refers to a protocol, where at least one critical reagent is withheld from the reaction mixture (or, if present in the reaction mixture, the reagent remains inactive) until the temperature is raised sufficiently to provide the necessary hybridization specificity of the primer or primers. A “hot start enzyme” is an enzyme, typically a nucleic acid polymerase, capable of acting as the “withheld” or inactive reagent in a hot start protocol. For example, some hot start enzymes can be obtained by chemically modifying the enzyme. Examples of hot-start enzymes include AZ05-Gold polymerase, KAPA HiFi and AmpliTaq Gold®.

As used herein, a “Taq extender” is a PCR additive that increases the efficiency of Taq DNA polymerase extension reactions during each cycle of PCR by reducing mismatch pausing, resulting in a greater percentage of completed extension reactions. In some embodiments, the Taq Extender comprises Taq Extender™ from Agilent or Strategene.

As used herein, the terms “individual”, “patient”, or “subject” can be an individual organism, a vertebrate, a mammal, or a human. In a preferred embodiment, the individual, patient or subject is a human.

A pseudogene is defined herein as a nucleic acid sequence which does not encode a wild type, functional, protein. The term “pseudogene” encompasses nucleic acid sequences which do not encode protein at all. Additionally, the term “pseudogene” encompasses gene alleles which comprise a modification, for instance an insertion or deletion so that they encode a protein or a part of a protein with significantly impaired, or lost, function as compared to a wild type protein of the same kind. Such allele for instance encodes a truncated protein as a result of a frame shift caused by an insertion and/or deletion of at least one nucleotide, or caused by a premature stop codon.

As used herein, the term “multiplex PCR” refers to the simultaneous generation of two or more PCR products or amplicons within the same reaction vessel. Each PCR product is primed using a distinct primer pair. A multiplex reaction may further include specific probes for each product that are labeled with different detectable moieties.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides that function as primers or probes are generally at least about 10-15 nucleotides in length or up to about 70, 100, 110, 150 or 200 nucleotides in length, and more preferably at least about 15 to 25 nucleotides in length. Oligonucleotides used as primers or probes for specifically amplifying or specifically detecting a particular target nucleic acid generally are capable of specifically hybridizing to the target nucleic acid.

A “positive control nucleic acid” or “internal positive amplification control” as used herein is a nucleic acid known to be present in a sample at a certain amount or level. In some embodiments, a positive control nucleic acid is not naturally present in a sample and is added to the sample prior to subjecting the reaction-sample mixture to real-time polymerase chain reaction in the disclosed methods.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

A “primer extension reaction” refers to a synthetic reaction in which an oligonucleotide primer hybridizes to a target nucleic acid and a complementary copy of the target nucleic acid is produced by the polymerase-dependent 3′-addition of individual complementary nucleotides. In some embodiments, the primer extension reaction is PCR.

As used herein, the phrase “primer pair” refers to a forward and reverse primer pair (i.e., a left and right primer pair) that can be used together to amplify a given region of a nucleic acid of interest.

As used herein, the phrase “gap primer pair” refers to primer pairs designed to produce a product only when a deletion is present. In some embodiments, the large deletions specifically detected using gap primer pairs include: a 7.6 Kb deletion in HEXA, a 6.4 Kb deletion in MCOLN1, a 2.5 Kb. Deletion in NEB, and the following alpha thalassemia variants: 3.7, 4.2, SEA, THAI, 20.5, MED and FIL. In some embodiments, detection of any of the 10 large deletions is based on the presence of gap PCR amplicons and not a particular variant being called

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a methylated target nucleic acid. Probes are typically at least about 10, 15, 20, 25, 30, 35, 40, 50, 60, 75, 100 nucleotides or more in length.

A “probe element” as used herein refers to a stretch of nucleotides that (a) is associated with a primer in that it is connected to or located adjacent to the primer nucleic acid sequence, and (b) specifically hybridizes under stringent conditions to a target nucleic acid sequence to be detected.

A “reaction-sample mixture” as used herein refers to a mixture containing amplification master mix and a sample.

As used herein, the term “sample” refers to clinical samples obtained from a patient or isolated microorganisms. In preferred embodiments, a sample is obtained from a biological source (i.e., a “biological sample”), such as tissue, bodily fluid, or microorganisms collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Preferred sample sources include nasopharyngeal and/or throat swabs or nasal washes.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%). Exemplary sensitivities include at least 50, 60, 70, 80, 90, 95, 98, and 99%.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity include at least 75%, at least 80%, at least 85%, at least 90%, at least 85-95%, at least 98%, at least 99%, or more sequence identity. Sequence identity can be determined using a commercially available computer program with a default setting that employs algorithms well known in the art. As used herein, sequences that have “high sequence identity” have identical nucleotides at least at about at least at about 75%, at least at about 80%, at least at about 85%, at least at about 90%, at least at about 95%, at least at about 99% or more of aligned nucleotide positions.

“Specificity,” as used herein, is a measure of the ability of a method to distinguish a truly occurring preselected sequence variant (gene variations) from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include at least 50, 60, 70, 80, 90, 95, 98, and 99%.

The term “stringent hybridization conditions” as used herein refers to hybridization conditions at least as stringent as the following: hybridization in 50% formamide, 5×SSC, 50 mM NaH₂PO4, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

The terms “target nucleic acid” or “target sequence” as used herein refer to a nucleic acid sequence of interest to be detected and/or quantified in the sample to be analyzed. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

### Methods for Detecting Gene Variants (Variations)

An aspect of the disclosure is directed to a method for detecting one or more variants for a genetic disorder in a subject that is suspected of being a carrier for at least one inherited genetic mutation comprising: extracting DNA from a biological sample obtained from the subject; generating a first plurality of amplicons by contacting the biological sample with a first plurality of primer pairs, wherein at least one amplicon corresponds to each of a plurality of genes, said plurality of genes comprising HEXA, SMPD1, MCOLN1, GBA, FANCC, IKBKAP, ASPA, BLM, BCKDHB, G6PC, ABCC8, DLD, NEB, PCDH15, CLRN1, TMEM216, and FKTN; generating a second plurality of amplicons by contacting the biological sample with a second plurality of primer pairs, wherein at least one amplicon corresponds to each of GBA and HBB, wherein the second plurality of primer pairs comprises SEQ ID NOs: 81 and 82, and SEQ ID NOs: 83 and 84; and generating a third plurality of amplicons by contacting the biological sample with a third plurality of primer pairs, a hotstart Taq DNA polymerase, and a Taq Extender PCR additive, wherein at least one amplicon corresponds to each of HBA1 and HBA2; incorporating a barcode sequence on to the ends of the first, second and third plurality of amplicons via a polymerase chain reaction; and detecting one or more variants in at least one of the first, second and third plurality of amplicons using high throughput massive parallel sequencing.

In some embodiments, the Ashkenazi Jewish panel variations comprise gene variations (e.g., single nucleotide variations (SNVs), deletions, insertions, or inversions) that are commonly observed in the Ashkenazi Jewish population, but that may also occur outside the Ashkenazi Jewish population as well, albeit in lower frequencies. In some embodiments, the Ashkenazi Jewish panel variations comprise genetic variations that can cause Tay-Sachs disease, Niemann Pick disease, Mucolipidosis Type IV, Gaucher disease, Fanconi Anemia, Familial Dysautonomia, Canavan disease, Bloom syndorme, Maple serum urinary disease, Glycogen storage disease I, Familial Hyperinsulinism, Dihydrolipoamide Dehydrogenase Deficiency (DLD), Lipoamide Dehydrogenase Deficiency (E3), Nemaline Myopathy, Usher Syndrome Type IF, Usher Syndrome, Type IIIA, Joubert Syndrome 2, or Walker-Warburg Syndrome. In some embodiments, the Ashkenazi Jewish panel variations comprise genetic variations listed in Table 1. In some embodiments, the beta thalassemia variations comprise genetic variations listed in Table 2. In some embodiments, the alpha thalassemia variations comprise genetic variations listed in Table 3.

In some embodiments, the first plurality of primer pairs comprises at least one gap primer pair designed to amplify target regions only when a deletion is present. In some embodiments, the GBA primers of the second plurality of primer pairs specifically amplify the GBA gene and not amplify any GBA pseudogenes. In some embodiments, the third plurality of primer pairs comprises at least one gap primer pair designed to amplify target regions only when a deletion is present.

In some embodiments, the HEXA gene variations cause Tay-Sachs disease; and the SMPD1 gene variations cause Niemann Pick disease; and the MCOLN1 gene variations cause Mucolipidosis Type IV; and the GBA gene variations cause Gaucher disease; and the PANCC gene variations cause Fanconi Anemia; and the IKBKAP gene variations cause Familial Dysautonomia; and the ASPA gene variations cause Canavan disease; and the BLM gene variations cause Bloom syndorme; and the BCKDHB gene variations cause Maple serum urinary disease; and the G6PC gene variations cause Glycogen storage disease I; and the ABCC8 gene variations cause Familial Hyperinsulinism; and the DLD gene variations cause Dihydrolipoamide Dehydrogenase Deficiency (DLD) or Lipoamide Dehydrogenase Deficiency (E3); and the NEB gene variations cause Nemaline Myopathy; and the PCDH15 gene variations cause Usher Syndrome, Type IF; and the CLRN1 gene variations cause Usher Syndrome, Type IIIA; and the TMEM216 gene variations cause Joubert Syndrome 2; and the FKTN gene variations cause Walker-Warburg Syndrome.

In some embodiments, the Ashkenazi Jewish panel gene variations comprise one or more gene variations recited in Table 1. In some embodiments, the HEXA gene variations comprise one or more of R178H (B1 variant), R247W, R249W, G269S, IVS9+1G>A, 1278_TATC, IVS12+1G>C, or 7.6-kb Del, Ex1; and the SMPD1 gene variations comprise one or more of L302P, fsP330, R496L (R498L), or deltaR608; and the MCOLN1 gene variations comprise one or more of IVS3−2A>G, or 6.4 kb_del; and the GBA gene variations comprise one or more of IVS2+1G>A, 84G>GG, N370S, del_55 bp, V394L, D409H, L444P, or R496H; and the PANCC gene variations comprise one or more of IVS4+4A>T, or 322delG; and the IKBKAP gene variations comprise one or more of R696P, or IVS20+6T>C; and the ASPA gene variations comprise one or more of IVS2−2A>G, Y231X, E285A, or A305E; and the BLM gene variations comprise one or more of 2281 del6/ins7; and the BCKDHB gene variations comprise one or more of R183P, G278S, or E372X; and the G6PC gene variations comprise one or more of R83C or Q347X; and the ABCC8 gene variations comprise IVS32−9G>A or F1387del; and the DLD gene variations comprise one or more of Y35* or G229C; and the NEB gene variations comprise R2478_D2512del35; and the PCDH15 gene variations comprise one or more of R245; and the CLRN1 gene variations comprise N48K; and the TMEM216 gene variations comprise R73L; and the FKTN gene variations comprise F390fs.

In some embodiments, the beta thalassemia gene variations in the HBB gene comprise one or more gene variations recited in Table 2. In some embodiments, the gene variations comprise c.*111A>G, c.*110T>C, c.*96T>C, Hb D-Los Angeles (c.364G>C), Hb O-Arab (c.364G>A), c.321_322insG, c.316−2A>C, c.316−2A>G, c.316−3C>A, c.316−106C>G, c.316−125A>G, c.316−146T>G, c.316−197C>T, c.315+1G>A, c.287_288insA, c.251 delG, c.230delC, c.216_217insA, c.203_204delTG, c.143_144insA, c.146_147insATCT, c.135delC, c.130G>T, c.126_129delCTTT, c.124_127delTTCT, c.118C>T, c.114G>A, c.112delT, c.93−1G>C, c.93−1G>A, c.93−21G>A, c.92+6T>C, c.92+5G>A, c.92+5G>C, c.92+5G>T, c.92+2T>A, c.92+2T>C, c.92+1G>A, c.92+1G>T, Hb Monroe (c.92G>C), c.92G>A, c.84_85insC, c.79G>T, HB E (c.79G>A), c.75T>A, c.59A>G, c.52A>T (LYS17*), c.51delC, c.48G>A, c.47G>A (Trp15), c.46delT, c.36delT, c.33C>A, c.27_28insG, c.25_26delAA, c.20delA, HB S (c.20A>T), HB C (c.19G>A), c.17_18delCT, c.2T>C, c.2T>G, c.1A>G, c.−78A>C, c.−78A>G, c.−79A>G, c.−80T>A, c.−81A>G, c.−136C>G, c.−137C>A, c.−137C>G, c.−137C>T, c.−138C>T, c.−138C>A, c.−140C>T, or c.−151C>T.

In some embodiments, the alpha thalassemia variations in the HBA1 and HBA2 genes comprise one or more variations recited in Table 3. In some embodiments, the alpha thalassemia variations in the HBA1 and HBA2 genes comprise large deletions selected from variant 3.7, variant 4.2, variant SEA, variant THAI, variant 20.5, variant MED or variant FIL; or a single nucleotide variant in the HBA2 gene HBA2 c.427T>C.

In some embodiments, the first plurality of primer pairs comprises at least two, at least four, at least five, at least eight or at least ten primer pairs having at least 85%, at least 88%, at least 90%, at least 93%, at least 95%, at least 99%, or more identity to at least two, at least four, at least five, at least eight or at least ten, at least 15, at least 20, at least 25, at least 30, at least 35, or all primer pairs selected from Table 4. In some embodiments, the first plurality of primer pairs comprises all the primer pairs shown in Table 4.

In some embodiments, the first plurality of primer pairs comprises at least two primer pairs having at least 85%, at least 88%, at least 90%, at least 93%, at least 95%, at least 99%, or more identity to at least two, at least three, at least four, at least five, at least 10, at least 15, at least 20, at least 25, at least 30 or all pairs selected from the group consisting of Pair 1 (SEQ ID NOs: 1-2), Pair 2 (SEQ ID NOs: 3-4), Pair 3 (SEQ ID NOs: 5-6), Pair 4 (SEQ ID NOs: 7-8), Pair 5 (SEQ ID NOs: 9-10), Pair 6 (SEQ ID NOs: 11-12), Pair 7 (SEQ ID NOs: 13-14), Pair 8 (SEQ ID NOs: 15-16), Pair 9 (SEQ ID NOs: 17-18), Pair 10 (SEQ ID NOs: 19-20), Pair 11 (SEQ ID NOs: 21-22), Pair 12 (SEQ ID NOs: 23-24), Pair 13 (SEQ ID NOs: 25-26), Pair 14 (SEQ ID NOs: 27-28), Pair 15 (SEQ ID NOs: 29-30), Pair 16 (SEQ ID NOs: 31-32), Pair 17 (SEQ ID NOs: 33-34), Pair 18 (SEQ ID NOs: 35-36), Pair 19 (SEQ ID NOs: 37-38), Pair 20 (SEQ ID NOs: 39-40), Pair 21 (SEQ ID NOs: 41-42), Pair 22 (SEQ ID NOs: 43-44), Pair 23 (SEQ ID NOs: 45-46), Pair 24 (SEQ ID NOs: 47-48), Pair 25 (SEQ ID NOs: 49-50), Pair 26 (SEQ ID NOs: 51-52), Pair 27 (SEQ ID NOs: 53-54), Pair 28 (SEQ ID NOs: 55-56), Pair 29 (SEQ ID NOs: 57-58), Pair 30 (SEQ ID NOs: 59-60), Pair 31 (SEQ ID NOs: 61-62), Pair 32 (SEQ ID NOs: 63-64), Pair 33 (SEQ ID NOs: 65-66), Pair 34 (SEQ ID NOs: 67-68), Pair 35 (SEQ ID NOs: 69-70), Pair 36 (SEQ ID NOs: 71-72), Pair 37 (SEQ ID NOs: 73-74), Pair 37 (SEQ ID NOs: 75-76), Pair 38 (SEQ ID NOs: 77-78), and Pair 40 (SEQ ID NOs: 79-80) of Table 4.

In some embodiments, the second plurality of primer pairs comprises at least two, at least four, or at least five primer pairs having at least 85%, at least 88%, at least 90%, at least 93%, at least 95%, at least 99%, or more identity to at least two, at least four, or at least five primer pairs selected from Table 5. In some embodiments, the first plurality of primer pairs comprises all the primer pairs shown in Table 5.

In some embodiments, the second plurality of primer pairs comprises at least two primer pairs having at least 85%, at least 88%, at least 90%, at least 93%, at least 95%, at least 99%, or more identity to at least two, at least four, or at least five primer primer pairs selected from SEQ ID NOs: 73 and 74, SEQ ID Nos: 75 and 76, SEQ ID NOs: 77 and 78, SEQ ID Nos: 79 and 80, SEQ ID NOs: 85 and 86, SEQ ID NOs: 87 and 88, or SEQ ID NOs: 89 and 90.

In some embodiments, the second plurality of primer pairs comprises at least two primer pairs having at least 85%, at least 88%, at least 90%, at least 93%, at least 95%, at least 99%, or more identity to at least two pairs selected from the group consisting of Pair 1 (SEQ ID NOs: 81-82), Pair 2 (SEQ ID NOs: 83-84), Pair 3 (SEQ ID NOs: 85-86), Pair 4 (SEQ ID NOs: 87-88), and Pair 5 (SEQ ID NOs: 89-90) of Table 6.

In some embodiments, the third plurality of primer pairs comprises at least two, at least four, at least five, at least eight or at least ten primer pairs having at least 85% identity to at least two, at least four, at least five, at least eight or at least ten primer pairs selected from Table 6. In some embodiments, the first plurality of primer pairs comprises all the primer pairs shown in Table 6.

In some embodiments, the third plurality of primer pairs comprises at least two primer pairs having at least 85%, at least 88%, at least 90%, at least 93%, at least 95%, at least 99%, or more identity to at least two pairs selected from the group consisting of Pair 1 (SEQ ID NOs: 91-92), Pair 2 (SEQ ID NOs: 93-94), Pair 3 (SEQ ID NOs: 95-96), Pair 4 (SEQ ID NOs: 97-98), Pair 5 (SEQ ID NOs: 99-100), Pair 6 (SEQ ID NOs: 101-102), Pair 7 (SEQ ID NOs: 103-104), and Pair 8 (SEQ ID NOs: 105-106), of Table 6.

In some embodiments, the next generation sequencing comprises sequencing by synthesis (e.g., pyrosequencing). In some embodiments, the next generation sequencing comprises sequencing by ligation.

In some embodiments, the next generation sequencing (aka. “high throughput, massively parallel sequencing”) employs sequencing-by-synthesis with reversible dye terminators. In other embodiments, sequencing is performed via sequencing-by-ligation. In yet other embodiments, sequencing is single molecule sequencing. Examples of next generation ssequencing techniques include, but are not limited to pyrosequencing, Reversible dye-terminator sequencing, SOLiD sequencing, Ion semiconductor sequencing, and Helioscope single molecule sequencing.

In some embodiments, the methods featured in the present technology are used in a multiplex, multi-gene assay format, e.g., assays that incorporate multiple signals from a large number of diverse genetic alterations in a large number of genes.

In some embodiments, the subject is of Ashkenazi Jewish descent or has a family history of, beta thalassemia or alpha thalassemia.

In some embodiments, the biological sample is selected from whole blood, serum, plasma, amniotic fluid or chorionic villi.

In some embodiments, the genetic disorder is an autosomal or X-linked recessive disorder.

### Methods for Detecting Inherited Mutations (e.g., Ashkenazi Jewish Panel Variations, Beta Thalassemia Variations or Alpha Thalassemia Variations) in a Single Assay

Upon preparing biological samples and next generation sequencing as described above, the present disclosure further provides methods for detecting the presence of inherited mutations (e.g., Ashkenazi Jewish panel variations, beta thalassemia variations or alpha thalassemia variations), present in target nucleic acid sequences amplified from a biological sample.

In some embodiments, the method further comprises determining whether the subject is a carrier for at least one of Ashkenazi Jewish panel variations, beta thalassemia variations or alpha thalassemia variations based on the next generation sequencing results. In some embodiments, the determining is achieved by: demultiplexing the sequencing reads; mapping the sequencing reads to a custom human reference genome wherein the pseudogene sequences within Reference Consortium human genome build 37(GRCh37) chr1: 155184031-155185327, chr1: 155188031-155188964, chr16: 223705-224240, and chr16: 227257-227413 are replaced with nucleotide T; filtering the mapped reads using a Phred Quality score requiring read mapping over 30; detecting variants in the filtered reads; and determining whether the subject is a carrier for Ashkenazi Jewish panel variations, beta thalassemia variations or alpha thalassemia variations.

In some embodiments, the subject is suspected of being a carrier for at least one disease selected from Tay-Sachs disease, Niemann Pick disease, Mucolipidosis Type IV, Gaucher disease, Fanconi Anemia, Familial Dysautonomia, Canavan disease, Bloom syndorme, Maple serum urinary disease, Glycogen storage disease I, Familial Hyperinsulinism, Dihydrolipoamide Dehydrogenase Deficiency (DLD), Lipoamide Dehydrogenase Deficiency (E3), Nemaline Myopathy, Usher Syndrome Type IF, Usher Syndrome, Type IIIA, Joubert Syndrome 2, Walker-Warburg Syndrome, beta thalassemia or alpha thalassemia.

### Kits

The present disclosure also provides kits for detecting the presence of Ashkenazi Jewish panel variations, beta thalassemia variations or alpha thalassemia variations in a biological sample from a subject.

Kits of the present technology comprise at least (i) a first plurality of primer pairs directed to amplifying regions of each of a plurality of genes, said plurality of genes comprising Hexosaminidase Subunit Alpha (HEXA), Sphingomyelin Phosphodiesterase 1 (SMPD1), Mucolipin TRP Cation Channel 1 (MCOLN1), Glucosylceramidase Beta (GBA), FA Complementation Group C (FANCC), Inhibitor of kappa light polypeptide gene enhancer in B-cells, kinase complex-associated protein (IKBKAP), Aspartoacylase (ASPA), Bloom Syndrome RecQ Like Helicase (BLM), Branched Chain Keto Acid Dehydrogenase E1 Subunit Beta (BCKDHB), Glucose-6-Phosphatase Catalytic Subunit (G6PC), ATP Binding Cassette Subfamily C Member 8 (ABCC8), Dihydrolipoamide Dehydrogenase (DLD), Nebulin (NEB), Protocadherin Related 15 (PCDH15), Clarin 1 (CLRN1), Transmembrane Protein 216 (TMEM216), and Fukutin (FKTN); (ii) a second plurality of primer pairs directed to amplifying regions of Glucosylceramidase Beta (GBA) and Hemoglobin Subunit Beta (HBB) genes, wherein the second plurality of primer pairs comprises SEQ ID NOs: 81 and 82, and SEQ ID NOs: 83 and 84; and (iii) a third plurality of primer pairs directed to amplifying regions of alpha thalassemia gene variations in each of Hemoglobin Subunit Alpha 1 (HBA1) and Hemoglobin Subunit Alpha 1 (HBA2) genes, and instructions for use.

In some embodiments, the first plurality of primer pairs comprises at least one gap primer pair designed to amplify target regions only when a deletion is present.

In some embodiments, the GBA primers of the second plurality of primer pairs specifically amplify the GBA gene and not amplify any GBA pseudogenes.

In some embodiments, the third plurality of primer pairs comprises at least one gap primer pair designed to amplify target regions only when a deletion is present.

In some embodiments, the HEXA gene variations cause Tay-Sachs disease; and the SMPD1 gene variations cause Niemann Pick disease; and the MCOLN1 gene variations cause Mucolipidosis Type IV; and the GBA gene variations cause Gaucher disease; and the PANCC gene variations cause Fanconi Anemia; and the IKBKAP gene variations cause Familial Dysautonomia; and the ASPA gene variations cause Canavan disease; and the BLM gene variations cause Bloom syndorme; and the BCKDHB gene variations cause Maple serum urinary disease; and the G6PC gene variations cause Glycogen storage disease I; and the ABCC8 gene variations cause Familial Hyperinsulinism; and the DLD gene variations cause Dihydrolipoamide Dehydrogenase Deficiency (DLD) or Lipoamide Dehydrogenase Deficiency (E3); and the NEB gene variations cause Nemaline Myopathy; and the PCDH15 gene variations cause Usher Syndrome, Type IF; and the CLRN1 gene variations cause Usher Syndrome, Type IIIA; and the TMEM216 gene variations cause Joubert Syndrome 2; and the FKTN gene variations cause Walker-Warburg Syndrome.

In some embodiments, the Ashkenazi Jewish panel gene variations comprise one or more gene variations recited in Table 1. In some embodiments, the HEXA gene variations comprise one or more of R178H (B1 variant), R247W, R249W, G269S, IVS9+1G>A, 1278_TATC, IVS12+1G>C, or 7.6-kb Del, Ex1; and the SMPD1 gene variations comprise one or more of L302P, fsP330, R496L (R498L), or deltaR608; and the MCOLN1 gene variations comprise one or more of IVS3−2A>G, or 6.4 kb_del; and the GBA gene variations comprise one or more of IVS2+1G>A, 84G>GG, N370S, del_55 bp, V394L, D409H, L444P, or R496H; and the PANCC gene variations comprise one or more of IVS4+4A>T, or 322delG; and the IKBKAP gene variations comprise one or more of R696P, or IVS20+6T>C; and the ASPA gene variations comprise one or more of IVS2-2A>G, Y231X, E285A, or A305E; and the BLM gene variations comprise one or more of 2281 del6/ins7; and the BCKDHB gene variations comprise one or more of R183P, G278S, or E372X; and the G6PC gene variations comprise one or more of R83C or Q347X; and the ABCC8 gene variations comprise IVS32−9G>A or F1387del; and the DLD gene variations comprise one or more of Y35* or G229C; and the NEB gene variations comprise R2478 D2512del35; and the PCDH15 gene variations comprise one or more of R245; and the CLRN1 gene variations comprise N48K; and the TMEM216 gene variations comprise R73L; and the FKTN gene variations comprise F390fs.

In some embodiments, the beta thalassemia gene variations in the HBB gene comprise one or more gene variations recited in Table 2. In some embodiments, the gene variations comprise c.*111A>G, c.*110T>C, c.*96T>C, Hb D-Los Angeles (c.364G>C), Hb O-Arab (c.364G>A), c.321_322insG, c.316−2A>C, c.316−2A>G, c.316−3C>A, c.316−106C>G, c.316−125A>G, c.316−146T>G, c.316−197C>T, c.315+1G>A, c.287_288insA, c.251delG, c.230delC, c.216_217insA, c.203_204delTG, c.143_144insA, c.146_147insATCT, c.135delC, c.130G>T, c.126_129delCTTT, c.124_127delTTCT, c.118C>T, c.114G>A, c.112delT, c.93−1G>C, c.93−1G>A, c.93−21G>A, c.92+6T>C, c.92+5G>A, c.92+5G>C, c.92+5G>T, c.92+2T>A, c.92+2T>C, c.92+1G>A, c.92+1G>T, Hb Monroe (c.92G>C), c.92G>A, c.84_85insC, c.79G>T, HB E (c.79G>A), c.75T>A, c.59A>G, c.52A>T (LYS17*), c.51delC, c.48G>A, c.47G>A (Trp15), c.46delT, c.36delT, c.33C>A, c.27_28insG, c.25_26delAA, c.20delA, HB S (c.20A>T), HB C (c.19G>A), c.17_18delCT, c.2T>C, c.2T>G, c.1A>G, c.−78A>C, c.−78A>G, c.−79A>G, c.−80T>A, c.−81A>G, c.−136C>G, c.−137C>A, c.−137C>G, c.−137C>T, c.−138C>T, c.−138C>A, c.−140C>T, or c.−151C>T.

In some embodiments, the alpha thalassemia variations in the HBA1 and HBA2 genes comprise one or more gene variations recited in Table 3. In some embodiments, the alpha thalassemia variations in the HBA1 and HBA2 genes comprise large deletions selected from variant 3.7, variant 4.2, variant SEA, variant THAI, variant 20.5, variant MED or variant FIL; or a single nucleotide variant in the HBA2 gene HBA2 c.427T>C.

In some embodiments, the first plurality of primer pairs comprises at least two, at least four, at least five, at least eight or at least ten primer pairs having at least 85%, at least 88%, at least 90%, at least 93%, at least 95%, at least 99%, or more identity to at least two, at least four, at least five, at least eight, at least 10, at least 15, at least 20, at least 25, at least 30, at least 35 or all primer pairs selected from Table 4. In some embodiments, the first plurality of primer pairs comprises all the primer pairs shown in Table 4.

In some embodiments, the first plurality of primer pairs comprises at least two primer pairs having at least 85%, at least 88%, at least 90%, at least 93%, at least 95%, at least 99%, or more identity to at least two, at least three, at least four, at least five, at least 10, at least 15, at least 20, at least 25, at least 30 or all pairs selected from the group consisting of Pair 1 (SEQ ID NOs: 1-2), Pair 2 (SEQ ID NOs: 3-4), Pair 3 (SEQ ID NOs: 5-6), Pair 4 (SEQ ID NOs: 7-8), Pair 5 (SEQ ID NOs: 9-10), Pair 6 (SEQ ID NOs: 11-12), Pair 7 (SEQ ID NOs: 13-14), Pair 8 (SEQ ID NOs: 15-16), Pair 9 (SEQ ID NOs: 17-18), Pair 10 (SEQ ID NOs: 19-20), Pair 11 (SEQ ID NOs: 21-22), Pair 12 (SEQ ID NOs: 23-24), Pair 13 (SEQ ID NOs: 25-26), Pair 14 (SEQ ID NOs: 27-28), Pair 15 (SEQ ID NOs: 29-30), Pair 16 (SEQ ID NOs: 31-32), Pair 17 (SEQ ID NOs: 33-34), Pair 18 (SEQ ID NOs: 35-36), Pair 19 (SEQ ID NOs: 37-38), Pair 20 (SEQ ID NOs: 39-40), Pair 21 (SEQ ID NOs: 41-42), Pair 22 (SEQ ID NOs: 43-44), Pair 23 (SEQ ID NOs: 45-46), Pair 24 (SEQ ID NOs: 47-48), Pair 25 (SEQ ID NOs: 49-50), Pair 26 (SEQ ID NOs: 51-52), Pair 27 (SEQ ID NOs: 53-54), Pair 28 (SEQ ID NOs: 55-56), Pair 29 (SEQ ID NOs: 57-58), Pair 30 (SEQ ID NOs: 59-60), Pair 31 (SEQ ID NOs: 61-62), Pair 32 (SEQ ID NOs: 63-64), Pair 33 (SEQ ID NOs: 65-66), Pair 34 (SEQ ID NOs: 67-68), Pair 35 (SEQ ID NOs: 69-70), Pair 36 (SEQ ID NOs: 71-72), Pair 37 (SEQ ID NOs: 73-74), Pair 37 (SEQ ID NOs: 75-76), Pair 38 (SEQ ID NOs: 77-78), and Pair 40 (SEQ ID NOs: 79-80) of Table 4.

In some embodiments, the second plurality of primer pairs comprises at least two, at least four, or at least five primer pairs having at least 85%, at least 88%, at least 90%, at least 93%, at least 95%, at least 99%, or more identity to at least two, at least four, or at least five primer pairs selected from Table 5. In some embodiments, the first plurality of primer pairs comprises all the primer pairs shown in Table 5.

In some embodiments, the second plurality of primer pairs comprises at least two primer pairs having at least 85%, at least 88%, at least 90%, at least 93%, at least 95%, at least 99%, or more identity to at least two, at least four, or at least five primer primer pairs selected from SEQ ID NOs: 73 and 74, SEQ ID Nos: 75 and 76, SEQ ID NOs: 77 and 78, SEQ ID Nos: 79 and 80, SEQ ID NOs: 85 and 86, SEQ ID NOs: 87 and 88, or SEQ ID NOs: 89 and 90.

In some embodiments, the second plurality of primer pairs comprises at least two primer pairs having at least 85%, at least 88%, at least 90%, at least 93%, at least 95%, at least 99%, or more identity to at least two pairs selected from the group consisting of Pair 1 (SEQ ID NOs: 81-82), Pair 2 (SEQ ID NOs: 83-84), Pair 3 (SEQ ID NOs: 85-86), Pair 4 (SEQ ID NOs: 87-88), and Pair 5 (SEQ ID NOs: 89-90) of Table 6.

In some embodiments, the third plurality of primer pairs comprises at least two, at least four, at least five, at least eight or at least ten primer pairs having at least 85% identity to at least two, at least four, at least five, at least eight or at least ten primer pairs selected from Table 6. In some embodiments, the first plurality of primer pairs comprises all the primer pairs shown in Table 6.

In some embodiments, the third plurality of primer pairs comprises at least two primer pairs having at least 85%, at least 88%, at least 90%, at least 93%, at least 95%, at least 99%, or more identity to at least two pairs selected from the group consisting of Pair 1 (SEQ ID NOs: 91-92), Pair 2 (SEQ ID NOs: 93-94), Pair 3 (SEQ ID NOs: 95-96), Pair 4 (SEQ ID NOs: 97-98), Pair 5 (SEQ ID NOs: 99-100), Pair 6 (SEQ ID NOs: 101-102), Pair 7 (SEQ ID NOs: 103-104), and Pair 8 (SEQ ID NOs: 105-106), of Table 6.

In some embodiments, the kits of the instant disclosure further comprise buffers, enzymes having polymerase activity, enzymes having polymerase activity and lacking 5′→3′ exonuclease activity or both 5′→3′ and 3′-»5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of alterations in target nucleic acid sequences corresponding to the specific set of inherited genetic mutations disclosed herein.

In some embodiments, the kits of the instant disclosure further comprise a positive control nucleic acid sequence and a negative control nucleic acid sequence to ensure the integrity of the assay during experimental runs. The kit may also comprise instructions for use, software for automated analysis, containers, packages such as packaging intended for commercial sale and the like.

The kits of the present technology can also include other necessary reagents to perform any of the NGS techniques disclosed herein. For example, the kit may further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

In some embodiments, the kits of the instant disclosure further comprise a plurality of barcoding primers selected from Pairs 1-4 of Table 7 or Pairs 1-9 of Table 8.

The kit may further comprise one or more of: wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kit additionally may comprise an assay definition scan card and/or instructions such as printed or electronic instructions for using the oligonucleotides in an assay. In some embodiments, a kit comprises an amplification reaction mixture or an amplification master mix. Reagents included in the kit may be contained in one or more containers, such as a vial.

Primers, probes, and/or primer-probes specific for amplification and detection of DNA internal control may be included in the amplification master mix as the target primer pairs to monitor potential PCR inhibition. Reagents necessary for amplification and detection of targets and internal control may be formulated as an all-in-one amplification master mix, which may be provided as single reaction aliquots in a kit.

### EXAMPLES

### Example 1: Sequencing Library Preparation and Raw Sequencing Data Generation

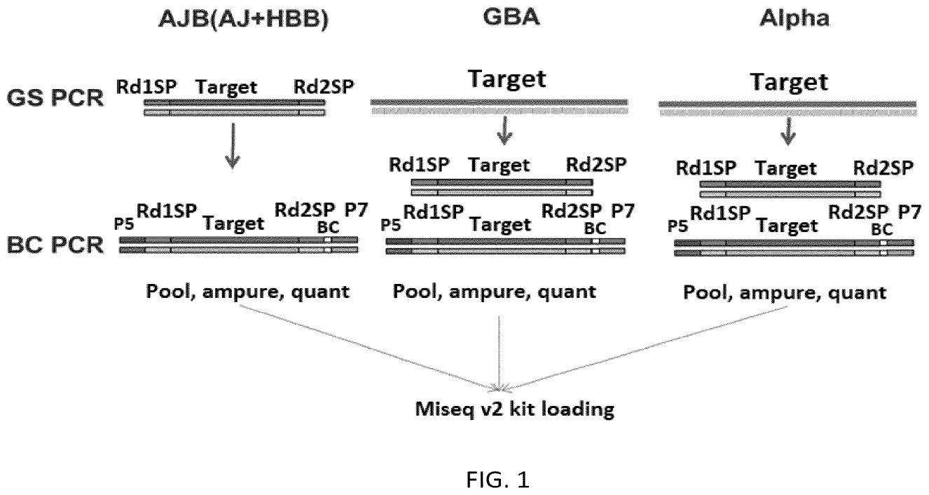

Three multiplex Gene Specific PCR reactions (GSP) were set up for each specimen to amplify target regions (FIG. 1). Each multiplex PCR reaction consists of a pool of primer pairs (See, Tables 4-6) that amplify various regions of genomic DNA containing the variations to be detected in this assay (See, Tables 1-3 for gene variations of interest). In total, fifty five amplicons were generated from the GSP reactions. After PCR, the GSP products for each patient were diluted 2500× with water. The diluted products were amplified with primers that add barcode sequences (BC) as well as the P5 and P7 adaptors (Table 7) needed for sequencing on the Illumina MiSeq. The barcoded samples were pooled and purified with Ampure XP beads and quantified. The final products were pooled into a single library and subjected to sequencing on the MiSeq. Sequencing data was analyzed by an in-house developed pipeline.

### Gene Specific PCR (GSP)

Three multiplex Gene Specific PCR reactions (GSP-A, -B, and -C) were set up for each specimen to amplify target regions. Reaction GSP-A contains 41 primer pairs, including 3 gap PCR primer pairs, to amplify target regions of 17 genes. The gap PCR primer pairs were designed to produce a product only when a deletion is present. The gene specific primer pairs were designed to contain Illumina sequencing primer binding sites, R1SP, for forward read and R2SP, for reverse and barcode read.

Reaction GSP-B contains 2 primer pairs for the GBA gene and 3 primer pairs for the HBB gene. GBA primers were designed to specifically amplify the GBA gene target regions while preventing the GBA pseudogene from co-amplification. Reaction GSP-C contains 16 primers for detection of 8 alpha thalassemia common variations and 1 amplification control. Gap PCR was employed for 7 alpha thalassemia common variations and thus, products are present only when deletions are present. In total, 55 amplicons were generated from the GSP reactions.

### Barcoding PCR (BCP)

To facilitate combining all the specimens simultaneously into a single library, each individual specimen had one of the 96 unique barcode sequences added. After gene specific PCR, the three GSP reactions for each patient were diluted 2500 fold with water and subjected to three barcoding PCR reactions (BCP-A, -B, and -C). During the barcoding PCR, the R1SP and R2SP sequences were used as priming sites to add barcode sequences as well as the P5 and P7 adapters that are needed for sequencing on the Illumina Miseq sequencer.

The barcoding PCR reaction B (GSP-B—see, Table 5) contains 5 primer pairs, and reaction C (GSP-C—Table 6) contains 16 primers, of R1SP and R2SP fused nested primers to generate 4 GBA specific nested PCR products and 8 alpha thalassemia nested PCR products, respectively.

In total, 57 amplicons were generated from the BCP reactions.

### Specimen Pooling and Ampure Cleanup

The barcoded samples were pooled into three separate tubes to create Pool-A, -B and -C and purified with Ampure XP beads followed by Qubit quantification. The final products were combined into a single library and subjected to sequencing on the MiSeq.

### Sequencing

The combined library was denatured prior to sequencing with 0.2N NaOH. The Illumina sequencing chemistry is based on sequencing by synthesis. The single-stranded library was loaded into the Miseq sequencing cartridge. Briefly, MiSeq workflow is as follows: the instrument begins by flushing the library through the flow cell where it hybridizes to the antisense P5 and P7 oligonucleotides that are complimentary to the adapters on the library. The library is diluted so that amplification generates well separated clusters of identical products from a single DNA molecule (clonal amplification). This is accomplished by isothermal bridge amplification. After cluster generation, the clusters are made single stranded and sequencing by synthesis begins. Fluorophore-labeled nucleotide triphosphates are applied to the flow cell. Nucleotides are 3′ blocked so that only a single nucleotide incorporates with each round of synthesis. The fluorophores are then excited by a laser and the emission spectra are recorded by the Miseq. The nucleotide blocker which had inhibited further synthesis is cleaved allowing for addition of the next nucleotide triphosphate. In this manner, fragments are sequenced.

### Example 2: Sequencing Data Analysis

### Alignment, Filtering and Variant Calling

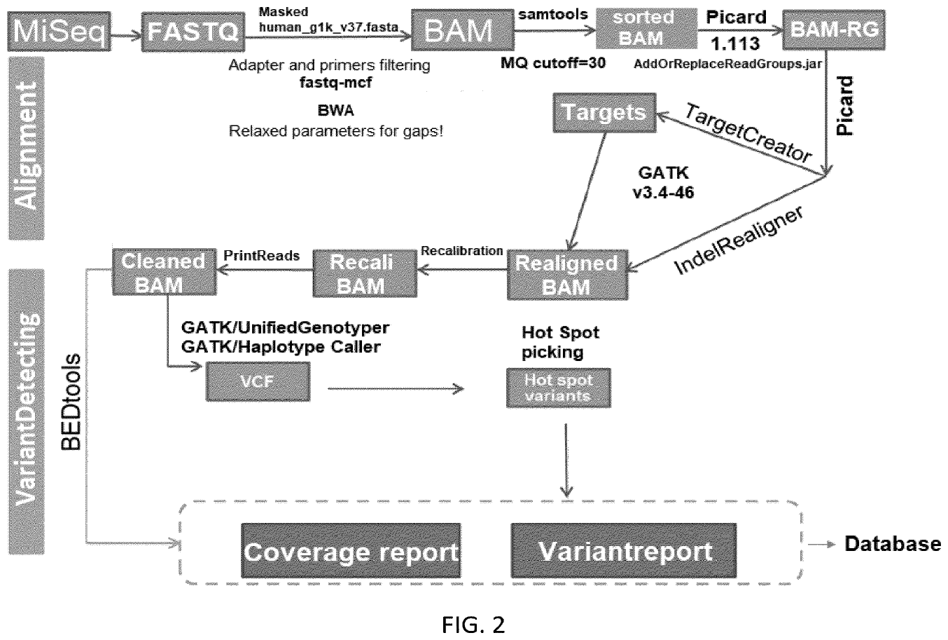

The data analysis pipeline workflow is shown in FIG. 2. De-multiplexed FASTQ files were generated using MiSeq Analysis software. The raw sequence reads in FASTQ files were then aligned to a custom reference genome using the Burrows-Wheeler Aligner (BWA). The custom reference genome differs from the Genome Reference Consortium human genome build 37(GRCh37) in that the highly homologous pseudogene sequence within chr1: 155184031-155185327, chr1: 155188031-155188964, chr16: 223705-224240, and chr16: 227257-227413 are replaced with nucleotide T for accurate alignment of GBA and for detection of the alpha thalassemia 4.2 deletion variant. Reads were then sorted and indexed using SAMtools followed by readgroup arrange using Picard Tools. Local realignment and base quality score recalibration were performed within targets using the Genome Analysis Toolkit (GATK). Mapped reads were further filtered using a Phred Quality score requiring read mapping over 30 (>99.9% accuracy), before downstream analysis. Average and minimum depth of coverage for every ROI (region of interest) were computed and variant calling is performed using GATK Unified and Haploid Genotyper callers. A single variant file (*.vcf) was created by merging variant files from both variant callers. Coverage and Variant Depth Reports were created and loaded to the sequencing database (seqDB). Alamut Batch was used to obtain high-level annotation for detected variants. Quality control rules for variant detection included the following: 1) must achieve minimum 40 reads in target position(s), 2) variant is detected at a frequency of 20% or higher. 3) Detection of large deletions was based on the presence of gap PCR amplicons with minimum 100 reads and not a particular variant being called.

### Example 3: Variants Detected

TABLE 1
Ashkenazi Jewish Panel

| | | | Conventional | | |
|---|---|---|---|---|---|
| # | Disorders | Gene | Name | Type | cDNA nomenclature |
| 1 | Tay-Sachs | HEXA | R178H (B1 variant) | SNV | c.532G > A |
| 2 | disease | | R247W | SNV | c.739C > T |
| 3 | | | R249W | SNV | c.745C > T |
| 4 | | | G269S | SNV | c.805G > A |
| 5 | | | IVS9 + 1G > A | SNV | c.1073 + 1G > A |
| 6 | | | 1278_TATC | INS | c.1274_1277dupTATC |
| 7 | | | IVS12 + 1G > C | SNV | c.1421 + 1G > C |
| 8 | | | 7.6-kb Del, Ex1 | LARGE | c.−2564_253 + 5128delinsG |
| | | | | DEL | |
| 9 | Niemann Pick | SMPD1 | L302P | SNV | c.911T > C |
| 10 | disease | | fsP330 | DEL | c.996delC |
| 11 | | | R496L (R498L) | SNV | c.1493G > T |
| 12 | | | deltaR608 | DEL | c.1829 1831del |
| | | | | | (g.6415770_6415772delGCC) |
| 13 | Mucolipidosis | MCOLN1 | IVS3-2A > G | SNV | c.406-2A > G |
| 14 | Type IV | | 6.4kb del | LARGE | 511del6.4kb (g.511_6943del) |
| | | | | DEL | |
| 15 | Gaucher disease | GBA | IVS2 + 1G > A | SNV | c.115 + 1G > A |
| 16 | | | 84G > GG | INS | 1035insG (c.84dupG) |
| 17 | | | N370S | SNV | c.1226A > G |
| 18 | | | del_55bp | DEL | c.1263de155 |
| 19 | | | V394L | SNV | c.1297G > T |
| 20 | | | D409H | SNV | c.1342G > C |
| 21 | | | L444P | SNV | c.1448T > C |
| 22 | | | R496H | SNV | c.1604G > A |
| 23 | Fanconi Anemia | FANCC | IVS4 + 4A > T | SNV | c.456 + 4A > T |
| 24 | | | 322delG | DEL | c.67delG |
| 25 | Familial | IKBKAP | R696P | SNV | c.2087G > C |
| | Dysautonomia | | | | |
| 26 | | | IVS20 + 6T > C | SNV | c.2204 + 6T > C |
| 27 | Canavan disease | ASPA | IVS2-2A > G | SNV | c.433-2A > G |
| 28 | | | Y231X | SNV | c.693C > A |
| 29 | | | E285A | SNV | c.854A > C |
| 30 | | | A305E | SNV | c.914C > A |
| 31 | Bloom | BLM | 2281del6/ins7 | INS/ | c.2207_2212del(ATCTGA) |
| | syndrome | | | DEL | insTAGATTC |
| 32 | Maple serum | BCKDHB | R183P | SNV | c.548G > C |
| | urinary disease | | | | |
| 33 | | | G278S | SNV | c.832G > A |
| 34 | | | E372X | SNV | c.1114G > T |
| 35 | Glycogen | G6PC | R83C | SNV | c.247C > T |
| | storage disease I | | | | |
| 36 | | | Q347X | SNV | c.1039C > T |
| 37 | Familial | ABCC8 | IVS32-9G > A | SNV | c.3989-9G > A |
| | Hyperinsulinism | | | | |
| 38 | | | F1387del | DEL | c.4160_4162delTCT |
| 39 | Dihydrolipoamide | DLD | Y35* | INS | c.104dupA |
| | Dehydrogenase | | | | |
| | Deficiency | | | | |
| | (DLD)/ | | | | |
| | Lipoamide | | | | |
| | Dehydrogenase | | | | |
| | Deficiency (E3) | | | | |
| 40 | | | G229C | SNV | c.685G > T |
| 41 | Nemaline | NEB | R2478_D2512del35 | LARGE | c.7432-2025_7536 + 372 |
| | Myopathy | | | DEL | del2502bp |
| 42 | Usher | PCDH15 | R245* | SNV | c.733C > T |
| | Syndrome, Type | | | | |
| | IF | | | | |
| 43 | Usher | CLRN1 | N48K | SNV | c.144T > G |
| | Syndrome, Type | | | | |
| | IIIA | | | | |
| 44 | Joubert | TMEM2 | R73L | SNV | c.218G > T |
| | Syndrome 2 | 16 | | | |
| 45 | Walker- | FKTN | F390fs | INS | c.1167dupA |
| | Warburg | | | | |
| | Syndrome | | | | |

TABLE 2
Beta Thalassemia related variants within HBB gene

| # | Name | Type | Location |
|---|---|---|---|
| 1 | c.*111A > G | SNV | 3′ TUR |
| 2 | c.*110T > C | SNV | 3′ TUR |
| 3 | c.*96T > C | SNV | 3′ TUR |
| 4 | Hb D-Los Angeles (c.364G > C) | SNV | Exon 3 |
| 5 | Hb O-Arab (c.364G > A) | SNV | Exon 3 |
| 6 | c.321_322insG | INS | Exon 3 |
| 7 | c.316-2A > C | SNV | IVS2 |
| 8 | c.316-2A > G | SNV | IVS2 |
| 9 | c.316-3C > A | SNV | IVS2 |
| 10 | c.316-106C > G | SNV | IVS2 |
| 11 | c.316-125A > G | SNV | IVS2 |
| 12 | c.316-146T > G | SNV | IVS2 |
| 13 | c.316-197C > T | SNV | IVS2 |
| 14 | c.315 + 1G > A | SNV | IVS2 |
| 15 | c.287_288insA | INS | Exon 2 |
| 16 | c.251delG | DEL | Exon 2 |
| 17 | c.230delC | DEL | Exon 2 |
| 18 | c.216_217insA | INS | Exon 2 |
| 19 | c.203_204delTG | DEL | Exon 2 |
| 20 | c.143_144insA | INS | Exon 2 |
| 21 | c.146_147insATCT | INS | Exon 2 |
| 22 | c.135delC | DEL | Exon 2 |
| 23 | c.130G > T | SNV | Exon 2 |
| 24 | c.126_129delCTTT | DEL | Exon 2 |
| 25 | c.124_127delTTCT | DEL | Exon 2 |
| 26 | c.118C > T | SNV | Exon 2 |
| 27 | c.114G > A | SNV | Exon 2 |
| 28 | c.112delT | DEL | Exon 2 |
| 29 | c.93-1G > C | SNV | IVS1 |
| 30 | c.93-1G > A | SNV | IVS1 |
| 31 | c.93-21G > A | SNV | IVS1 |
| 32 | c.92 + 6T > C | SNV | IVS1 |
| 33 | c.92 + 5G > A | SNV | IVS1 |
| 34 | c.92 + 5G > C | SNV | IVS1 |
| 35 | c.92 + 5G > T | SNV | IVS1 |
| 36 | c.92 + 2T > A | SNV | IVS1 |
| 37 | c.92 + 2T > C | SNV | IVS1 |
| 38 | c.92 + 1G > A | SNV | IVS1 |
| 39 | c.92 + 1G > T | SNV | IVS1 |
| 40 | Hb Monroe (c.92G > C) | SNV | Exon 1 |
| 41 | c.92G > A | SNV | Exon 1 |
| 42 | c.84_85insC | INS | Exon 1 |
| 43 | c.79G > T | SNV | Exon 1 |
| 44 | HB E (c.79G > A) | SNV | Exon 1 |
| 45 | c.75T > A | SNV | Exon 1 |
| 46 | c.59A > G | SNV | Exon 1 |
| 47 | c.52A > T (LYS17*) | SNV | Exon 1 |
| 48 | c.51delC | DEL | Exon 1 |
| 49 | c.48G > A | SNV | Exon 1 |
| 50 | c.47G > A (Trp15) | SNV | Exon 1 |
| 51 | c.46delT | DEL | Exon 1 |
| 52 | c.36delT | DEL | Exon 1 |
| 53 | c.33C > A | SNV | Exon 1 |
| 54 | c.27_28insG | INS | Exon 1 |
| 55 | c.25_26delAA | DEL | Exon 1 |
| 56 | c.20delA | DEL | Exon 1 |
| 57 | HB S (c.20A > T) | SNV | Exon 1 |
| 58 | HB C (c.19G > A) | SNV | Exon 1 |
| 59 | c.17_18delCT | DEL | Exon 1 |
| 60 | c.2T > C | SNV | Exon 1 |
| 61 | c.2T > G | SNV | Exon 1 |
| 62 | c.1A > G | SNV | Exon 1 |
| 63 | c.−78A > C | SNV | 5′ UTR |
| 64 | c.−78A > G | SNV | 5′ UTR |
| 65 | c.−79A > G | SNV | 5′ UTR |
| 66 | c.−80T > A | SNV | 5′ UTR |
| 67 | c.−81A > G | SNV | 5′ UTR |
| 68 | c.−136C > G | SNV | 5′ UTR |
| 69 | c.−137C > A | SNV | 5′ UTR |
| 70 | c.−137C > G | SNV | 5′ UTR |
| 71 | c.−137C > T | SNV | 5′ UTR |
| 72 | c.−138C > T | SNV | 5′ UTR |
| 73 | c.−138C > A | SNV | 5′ UTR |
| 74 | c.−140C > T | SNV | 5′ UTR |
| 75 | c.−151C > T | SNV | 5′ UTR |

TABLE 3
Alpha Thalassemia related variants

| # | Variant | Type | Gene |
|---|---|---|---|
| 1 | 3.7 | Large Deletion | HBA1 and HBA2 |
| 2 | 4.2 | Large Deletion | HBA2 |
| 3 | SEA | Large Deletion | HBA1 and HBA2 |
| 4 | THAI | Large Deletion | HBA1 and HBA2 |
| 5 | 20.5 | Large Deletion | HBA1 and HBA2 |
| 6 | MED | Large Deletion | HBA1 and HBA2 |
| 7 | FIL | Large Deletion | HBA1 and HBA2 |
| 8 | Constant Spring | SNV | HBA2 c.427T > C |

### Example 4: Primers and Other Materials

TABLE 4
GSP-A primers

| | | | SEQ |
|---|---|---|---|
| Pair | Name | Sequence | ID NO |
| 1 | HEX-101-F | ACACTCTTTCCCTACACGACGCTCTTCCGATCT TGACATGTTCAATGTTTGTTCTGC | 1 |
| | HEX-101-R | GTGACTGGAGTTCAGACGTGTGCTCTTCCGATCT GTCTGTCCGTTGCTCCATC | 2 |
| 2 | HEX-102-F | ACACTCTTTCCCTACACGACGCTCTTCCGATCT CAACCCTGTCACCCACATC | 3 |
| | HEX-102-R | GTGACTGGAGTTCAGACGTGTGCTCTTCCGATCT TGAGCATAACAAGCAGAGTCC | 4 |
| 3 | HEX-103-F | ACACTCTTTCCCTACACGACGCTCTTCCGATCT TTGGGATTCAGGAAGTCCAAC | 5 |
| | HEX-103-R | GTGACTGGAGTTCAGACGTGTGCTCTTCCGATCT ACCAGACAGTGGCCAAG | 6 |
| 4 | HEX-104-F2 | ACACTCTTTCCCTACACGACGCTCTTCCGATCT CCAGACACAATCATACAGGTGTG | 7 |
| | HEX-104-R | GTGACTGGAGTTCAGACGTGTGCTCTTCCGATCT CCAGCCTCCTTTGGTTAGCAAG | 8 |
| 5 | HEX-105-F | ACACTCTTTCCCTACACGACGCTCTTCCGATCT ATCACCAGACTGTTGTTGCTTG | 9 |
| | HEX-105-R | GTGACTGGAGTTCAGACGTGTGCTCTTCCGATCT CTAGTCCAGAGGTGGCTAGATG | 10 |
| 6 | HEX-106c-F | ACACTCTTTCCCTACACGACGCTCTTCCGATCT TAGGACCCTGGCTAGCTTGA | 11 |
| | HEX-106c-R | GTGACTGGAGTTCAGACGTGTGCTCTTCCGATCT CTCTGATCACAGGCACTCCA | 12 |
| 7 | HEX-106-F | ACACTCTTTCCCTACACGACGCTCTTCCGATCT TACCTGAGATGTTTTGATATAGGCATCC | 13 |
| | HEX-106-R | GTGACTGGAGTTCAGACGTGTGCTCTTCCGATCT GACCCCCATCTCTACAAAATGCA | 14 |
| 8 | SMP-107-F4 | ACACTCTTTCCCTACACGACGCTCTTCCGATCT ACATGATGTCTGGCACCAGA | 15 |
| | SMP-107-R3 | GTGACTGGAGTTCAGACGTGTGCTCTTCCGATCT CTTGGCCATCGCTTCATAGA | 16 |
| 9 | SMP-108-F | ACACTCTTTCCCTACACGACGCTCTTCCGATCT CAGAGCCTGCAAAGCATGG | 17 |
| | SMP-108-R | GTGACTGGAGTTCAGACGTGTGCTCTTCCGATCT ATGTTTGCCTGGGTCAGATTCA | 18 |
| 10 | SMP-109-F | ACACTCTTTCCCTACACGACGCTCTTCCGATCT CTGGTTTCTCTACCATAAGGGCC | 19 |
| | SMP-109-R | GTGACTGGAGTTCAGACGTGTGCTCTTCCGATCT CATCAAGAACTTTCCCAAATGTGGG | 20 |
| 11 | MCO-110-F | ACACTCTTTCCCTACACGACGCTCTTCCGATCT TGCTGGTGGGCAGGCAGGT | 21 |
| | MCO-110-R | GTGACTGGAGTTCAGACGTGTGCTCTTCCGATCT GAGCAAGCCCTGAGCCATT | 22 |
| 12 | MCO-111c-F | ACACTCTTTCCCTACACGACGCTCTTCCGATCT CTGGGATTTGATGCTAGGG | 23 |
| | MCO-111c-R | GTGACTGGAGTTCAGACGTGTGCTCTTCCGATCT TTATAACCTGGCCCATCAGC | 24 |
| 13 | MCO-111-F | ACACTCTTTCCCTACACGACGCTCTTCCGATCT CAGTACTGTTTGCCCAGCCT | 25 |
| | MCO-111-R | GTGACTGGAGTTCAGACGTGTGCTCTTCCGATCT CTCACCGTGCTGGAAGACACTG | 26 |
| 14 | FAN-116-F | ACACTCTTTCCCTACACGACGCTCTTCCGATCT TTTAGGGTGTATTATCTCATATACTTTCAG | 27 |
| | FAN-116-R | GTGACTGGAGTTCAGACGTGTGCTCTTCCGATCT ATCTCTTCTGGAGGACTGAAATATTTCC | 28 |
| 15 | FAN-117-F | ACACTCTTTCCCTACACGACGCTCTTCCGATCT CTGCTGTGAAGGGACATCAC | 29 |
| | FAN-117-R | GTGACTGGAGTTCAGACGTGTGCTCTTCCGATCT CTCTTTCAAGGCTTCATACATCTTCC | 30 |
| 16 | IKB-118-F | ACACTCTTTCCCTACACGACGCTCTTCCGATCT ATGGTGTGTTTAGCATTACAGGC | 31 |
| | IKB-118-R | GTGACTGGAGTTCAGACGTGTGCTCTTCCGATCT CAAGAATTATGCTTGGTACTTGGCT | 32 |
| 17 | IKB-119-F | ACACTCTTTCCCTACACGACGCTCTTCCGATCT ATGCCAAGGGGAAACTTAGAAGTTGTTC | 33 |
| | IKB-119-R | GTGACTGGAGTTCAGACGTGTGCTCTTCCGATCT GCCTCTAAGAATCTGATTGATGATATA | 34 |
| 18 | ASP-120-F | ACACTCTTTCCCTACACGACGCTCTTCCGATCT CCAATCTTAGTTGATGAAGTAAAACGTATTG | 35 |
| | ASP-120-R | GTGACTGGAGTTCAGACGTGTGCTCTTCCGATCT TTGGCTATGGAACGAGTGGTC | 36 |
| 19 | ASP-121-F2 | ACACTCTTTCCCTACACGACGCTCTTCCGATCT TGTCATAGGAAAAGAATTTCCTCCC | 37 |
| | ASP-121-R2 | GTGACTGGAGTTCAGACGTGTGCTCTTCCGATCT GTGCTGTATGAGCTATAAACTTCTATTC | 38 |
| 20 | ASP-122-F | ACACTCTTTCCCTACACGACGCTCTTCCGATCT GATGGGAAGACGATCCCACTG | 39 |
| | ASP-122-R2 | GTGACTGGAGTTCAGACGTGTGCTCTTCCGATCT GTAAGACACCGTGTAAGATGTAAGC | 40 |
| 21 | BLM-123-F | ACACTCTTTCCCTACACGACGCTCTTCCGATCT CCTTTGATAGGTTTGATATGTGACTAA | 41 |
| | BLM-123-R | GTGACTGGAGTTCAGACGTGTGCTCTTCCGATCT CCTTTTCTGGAGTGACATATAGAAGTT | 42 |
| 22 | BCK-124-F | ACACTCTTTCCCTACACGACGCTCTTCCGATCT GAAGGACTCATTGTGCCATGC | 43 |
| | BCK-124-R2 | GTGACTGGAGTTCAGACGTGTGCTCTTCCGATCT TGAACATACCTTGATTCCTGGGCAA | 44 |
| 23 | BCK-125-F2 | ACACTCTTTCCCTACACGACGCTCTTCCGATCT CTGTATTTAGCGGAAGAAGTCCCTA | 45 |
| | BCK-125-R | GTGACTGGAGTTCAGACGTGTGCTCTTCCGATCT TGTATCAGGATTTAAAATGAGAGCTTCC | 46 |
| 24 | BCK-126-F | ACACTCTTTCCCTACACGACGCTCTTCCGATCT TGACTCTGTCTGCAGGAGG | 47 |
| | BCK-126-R | GTGACTGGAGTTCAGACGTGTGCTCTTCCGATCT GTCAGGGGCACATAGTAGCTT | 48 |
| 25 | G6P-127-F | ACACTCTTTCCCTACACGACGCTCTTCCGATCT AGGCTACACTCTTCTTGAAGGTG | 49 |
| | G6P-127-R | GTGACTGGAGTTCAGACGTGTGCTCTTCCGATCT CTGGTCCAGTCTCACAGGTT | 50 |
| 26 | G6P-128-F2 | ACACTCTTTCCCTACACGACGCTCTTCCGATCT GTCTTCTACGTCTTGTCCTTCTGCAAG | 51 |
| | G6P-128-R | GTGACTGGAGTTCAGACGTGTGCTCTTCCGATCT CATTGCTTCAAATAGTAGTCCTCC | 52 |
| 27 | ABC-201-F | ACACTCTTTCCCTACACGACGCTCTTCCGATCT TCCAAGGAGGAGTGTGTCTG | 53 |
| | ABC-201-R | GTGACTGGAGTTCAGACGTGTGCTCTTCCGATCT TGCTTCAGCACCGGCTTCAG | 54 |
| 28 | ABC-202-F | ACACTCTTTCCCTACACGACGCTCTTCCGATCT CATAGAGGCTATTCCCAGCAGC | 55 |
| | ABC-202-R | GTGACTGGAGTTCAGACGTGTGCTCTTCCGATCT CATCTGGTGGCTGTGGGTAC | 56 |
| 29 | DLD-203-F | ACACTCTTTCCCTACACGACGCTCTTCCGATCT GAGCTTCTCATAGGAACATACTAGCG | 57 |
| | DLD-203-R | GTGACTGGAGTTCAGACGTGTGCTCTTCCGATCT TAGATATCTCCATATCAATTCCAACTCC | 58 |
| 30 | DLD-204-F | ACACTCTTTCCCTACACGACGCTCTTCCGATCT GTTATCATCAACCTTCAGTAGTTCTATGTTTTGA | 59 |
| | DLD-204-R | GTGACTGGAGTTCAGACGTGTGCTCTTCCGATCT TTTTACATTGAAATAGAAAGGAACTGTCAGCTA | 60 |
| 31 | NEB-205c-F | ACACTCTTTCCCTACACGACGCTCTTCCGATCT AGCCACTGTGTCCAGCTTCT | 61 |
| | NEB-205c-R | GTGACTGGAGTTCAGACGTGTGCTCTTCCGATCT TTGGATCCCCTTGTCTTCTG | 62 |
| 32 | NEB-205-F2 | ACACTCTTTCCCTACACGACGCTCTTCCGATCT ATGAGATTCTCCTGCGTGGGT | 63 |
| | NEB-205-R2 | GTGACTGGAGTTCAGACGTGTGCTCTTCCGATCT GGTAGTGCAGAACTGGGATACACAC | 64 |
| 33 | PCD-206-F | ACACTCTTTCCCTACACGACGCTCTTCCGATCT AAGTTCTGGGATTACAGGTATGG | 65 |
| | PCD-206-R | GTGACTGGAGTTCAGACGTGTGCTCTTCCGATCT ATCACGAGTGTTTGGCACAAGG | 66 |
| 34 | CLR-207-F | ACACTCTTTCCCTACACGACGCTCTTCCGATCT AGTTGTGACAGCCTTGGGGACA | 67 |
| | CLR-207-R | GTGACTGGAGTTCAGACGTGTGCTCTTCCGATCT TGCAATTGCTACTTACATGAGAACCG | 68 |
| 35 | TME-208-F | ACACTCTTTCCCTACACGACGCTCTTCCGATCT GCTGTTATATGCTGTTTGCAAACTC | 69 |
| | TME-208-R | GTGACTGGAGTTCAGACGTGTGCTCTTCCGATCT TGTTAATCAAGTTACAGATACCAGC | 70 |
| 36 | FKT-209-F | ACACTCTTTCCCTACACGACGCTCTTCCGATCT AAGAAACTGATCACATGTGGAATGGA | 71 |
| | FKT-209-R2 | GTGACTGGAGTTCAGACGTGTGCTCTTCCGATCT GTGTTAGCTAATAATGGAGTCTTGAAAGAGA | 72 |
| 37 | BetaE1-F | ACACTCTTTCCCTACACGACGCTCTTCCGATCT GTCTCCTTAAACCTGTCTTGTA | 73 |
| | BetaE1-R | GTGACTGGAGTTCAGACGTGTGCTCTTCCGATCT CCAGTGCCAGAAGAGCCAAG | 74 |
| 38 | BetaE2-F | ACACTCTTTCCCTACACGACGCTCTTCCGATCT GGGAAAGAAAACATCAAGCGTCCCAT | 75 |
| | BetaE2-R | GTGACTGGAGTTCAGACGTGTGCTCTTCCGATCT GACAGAGAAGACTCTTGGGTTTCTG | 76 |
| 39 | BetaE3-F | ACACTCTTTCCCTACACGACGCTCTTCCGATCT ACTGACCTCCCACATTCCCTTT | 77 |
| | BetaE3-R | GTGACTGGAGTTCAGACGTGTGCTCTTCCGATCT TCATGTTCATACCTCTTATCTTCC | 78 |
| 40 | BetalVS2-F | ACACTCTTTCCCTACACGACGCTCTTCCGATCT TAGCTTGGACTCAGAATAATCCA | 79 |
| | BetalVS2-R | GTGACTGGAGTTCAGACGTGTGCTCTTCCGATCT CCTAATCTCTTTCTTTCAGGGCA | 80 |

TABLE 5
GSP-B primers

| | | | SEQ |
|---|---|---|---|
| Pair | Name | Sequence | ID NO |
| 1 | GDL1-for | AAAGTTGTCACCCATACATGCCCT | 81 |
| | GDL1-rev | GGAAGCTGAAGCAAGAGAATCGC | 82 |
| 2 | GDL2-for | CAAAGCAGACCTCAGACCTCTTAC | 83 |
| | GDL2-rev | CTGGGCTTACGTCGCTGTAA | 84 |
| 3 | BetaE1-Fb | ACACTCTTTCCCTACACGACGCTCTTCCGATCT catgcccagtttctattggt | 85 |
| | BetaE1-Rb | GTGACTGGAGTTCAGACGTGTGCTCTTCCGATCT ggtttgaagtccaactccta | 86 |
| 4 | BetaE2-Fb | ACACTCTTTCCCTACACGACGCTCTTCCGATCT agaaggggaaagaaaacatcaag | 87 |
| | BetaE2-Rb | GTGACTGGAGTTCAGACGTGTGCTCTTCCGATCT GACAGAGAAGACTCTTGGGTTTCTG | 88 |
| 5 | BetaE3-Fb | ACACTCTTTCCCTACACGACGCTCTTCCGATCT ccaaggtttgaactagctc | 89 |
| | BetaE3-Rb | GTGACTGGAGTTCAGACGTGTGCTCTTCCGATCT ggattattctgagtccaagc | 90 |

TABLE 6
GSP-C primers

| | | | SEQ |
|---|---|---|---|
| Pair | Name | Sequence | ID NO |
| 1 | a2/3.7-F | CCCCTCGCCAAGTCCACCC | 91 |
| | a2-R | AGACCAGGAAGGGCCGGTG | 92 |
| 2 | 4.2-F | GGTTTACCCATGTGGTGCCTC | 93 |
| | 4.2-R | CCCGTTGGATCTTCTCATTTCCC | 94 |
| 3 | 3.7/20.5-R | AAAGCACTCTAGGGTCCAGCG | 95 |
| | 20.5-F2 | GCACAAATTTCTAGGATGAGTGTGG | 96 |
| 4 | SEA-F | CGATCTGGGCTCTGTGTTCTC | 97 |
| | SEA-R | AGCCCACGTTGTGTTCATGGC | 98 |
| 5 | MED-F | TACCCTTTGCAAGCACACGTAC | 99 |
| | MED-R | TCAATCTCCGACAGCTCCGAC | 100 |
| 6 | THAI-F | GACCATTCCTCAGCGTGGGTG | 101 |
| | THAI-R | CAAGTGGGCTGAGCCCTTGAG | 102 |
| 7 | FIL-F2 | TTTAAATGGGCAAAACAGGCCAGG | 103 |
| | FIL-R2 | ATAACCTTTATCTGCCACATGTAGC | 104 |
| 8 | LIS1-F | GTCGTCACTGGCAGCGTAGATC | 105 |
| | LIS1-R | GATTCCAGGTTGTAGACGGACTG | 106 |

TABLE 7
BCB-B primers

| | | | SEQ |
|---|---|---|---|
| Pair | Name | Sequence | ID NO |
| 1 | GBA-112-F | ACACTCTTTCCCTACACGACGCTCTTCCGATCT CAGTCATTCCTCATTCTGTCCTC | 107 |
| | GBA-112-R | GTGACTGGAGTTCAGACGTGTGCTCTTCCGATCT TACCACCAGCTTACTGGAAGG | 108 |
| 2 | GBA-113-F | ACACTCTTTCCCTACACGACGCTCTTCCGATCT TTGCCTTTGTCCTTACCCTAG | 109 |
| | GBA-113-R | GTGACTGGAGTTCAGACGTGTGCTCTTCCGATCT ACATGGGCTGTTTGTAAAACGTG | 110 |
| 3 | GBA-114-F | ACACTCTTTCCCTACACGACGCTCTTCCGATCT GACTTCTTAGATGAGGGTTTCATG | 111 |
| | GBA-114-R | GTGACTGGAGTTCAGACGTGTGCTCTTCCGATCT GCTGTCTTCAGCCCACTTC | 112 |
| 4 | GBA-115-F | ACACTCTTTCCCTACACGACGCTCTTCCGATCT CAGGATCACACTCTCAGCTTCTCC | 113 |
| | GBA-115-R | GTGACTGGAGTTCAGACGTGTGCTCTTCCGATCT CAGCGTGTGAGCTGACTCTGTC | 114 |

TABLE 8
BCP-C primers

| | | | SEQ |
|---|---|---|---|
| Pair | Name | Sequence | ID NO |
| 1 | 3.7nest-F | ACACTCTTTCCCTACACGACGCTCTTCCGATCT CTTTGAATAAAGTCTGAGTGGGC | 115 |
| | 3.7/20.5nest-R | GTGACTGGAGTTCAGACGTGTGCTCTTCCGATCT AAAGCACTCTAGGGTCCAGCG | 116 |
| 2 | 4.2nest-F | ACACTCTTTCCCTACACGACGCTCTTCCGATCT CAGACCAAGGACCTCTCTGC | 117 |
| | 4.2nest-R | GTGACTGGAGTTCAGACGTGTGCTCTTCCGATCT GAAGTGTGAGTAGGGCCAGC | 118 |
| 3 | SEAnest-F | ACACTCTTTCCCTACACGACGCTCTTCCGATCT CGATCTGGGCTCTGTGTTCTC | 119 |
| | SEAnest-R2 | GTGACTGGAGTTCAGACGTGTGCTCTTCCGATCT CTCCAGAACATTTGGCGATT | 120 |
| 4 | THAInest-F | ACACTCTTTCCCTACACGACGCTCTTCCGATCT TGGCACCTACCCTGGAAACA | 121 |
| | THAInest-R | GTGACTGGAGTTCAGACGTGTGCTCTTCCGATCT AGGGAAGGCAAGTCCAGAGG | 122 |
| 5 | 20.5nest-F | ACACTCTTTCCCTACACGACGCTCTTCCGATCT GCACAAATTTCTAGGATGAGTGTGG | 123 |
| | 20.5nest-R | GTGACTGGAGTTCAGACGTGTGCTCTTCCGATCT CATGTACTCCGGATGTTGGGC | 124 |
| 6 | MEDnest-F | ACACTCTTTCCCTACACGACGCTCTTCCGATCT TACCCTTTGCAAGCACACGTAC | 125 |
| | MEDnest-R | GTGACTGGAGTTCAGACGTGTGCTCTTCCGATCT GAAGGTGAGGTCATGCAGGC | 126 |
| 7 | FILnest-F | ACACTCTTTCCCTACACGACGCTCTTCCGATCT TTTAAATGGGCAAAACAGGCCAGG | 127 |
| | FILnest-R | GTGACTGGAGTTCAGACGTGTGCTCTTCCGATCT ATAACCTTTATCTGCCACATGTAGC | 128 |
| 8 | Alnest-F6 | ACACTCTTTCCCTACACGACGCTCTTCCGATCT GGCGAGATGGCGCCTTCCTC | 129 |
| | a2nest-R | GTGACTGGAGTTCAGACGTGTGCTCTTCCGATCT AGACCAGGAAGGGCCGGTG | 130 |
| 9 | LISnest-F | ACACTCTTTCCCTACACGACGCTCTTCCGATCT AGCATGAATTGTTTAAGGGTAAGC | 131 |
| | LISnest-R | GTGACTGGAGTTCAGACGTGTGCTCTTCCGATCT AACTCAACTCAGCAGCAGGCTGT | 132 |

TABLE 9
GSP-A primer pool

| Reagent | GSP-A primer pool (3X) | | | | | | |
|---|---|---|---|---|---|---|---|
| Storage | Store at −10° C. or colder. | | | | | | |
| Stability | Stable for 1 year. Avoid freeze/thaw cycles. | | | | | | |
| | | Stock | | | | | Final |
| | GSP-A | conc. | ul/rxn | | ul/4000 rxn | | conc. |
| | primer | (uM) | F | R | F | R | (uM) |
| Preparation | HEX-101 | 100 | 0.030 | 0.030 | 120 | 120 | 0.20 |
| | HEX-102 | 100 | 0.011 | 0.011 | 45 | 45 | 0.08 |
| | HEX-103 | 100 | 0.030 | 0.030 | 120 | 120 | 0.20 |
| | HEX-104 | 100 | 0.030 | 0.030 | 120 | 120 | 0.20 |
| | HEX-105 | 100 | 0.030 | 0.030 | 120 | 120 | 0.20 |
| | HEX-106c | 100 | 0.015 | 0.015 | 60 | 60 | 0.10 |
| | HEX-106 | 100 | 0.090 | 0.090 | 360 | 360 | 0.60 |
| | SMP-107 | 100 | 0.030 | 0.030 | 120 | 120 | 0.20 |
| | SMP-108 | 100 | 0.030 | 0.030 | 120 | 120 | 0.20 |
| | SMP-109 | 100 | 0.030 | 0.030 | 120 | 120 | 0.20 |
| | MCO-110 | 1000 | 0.015 | 0.015 | 60 | 60 | 1.00 |
| | MCO-111c | 100 | 0.030 | 0.030 | 120 | 120 | 0.20 |
| | MCO-111 | 100 | 0.090 | 0.090 | 360 | 360 | 0.60 |
| | FAN-116 | 1000 | 0.030 | 0.030 | 120 | 120 | 2.00 |
| | FAN-117 | 100 | 0.030 | 0.030 | 120 | 120 | 0.20 |
| | IKB-118 | 100 | 0.030 | 0.030 | 120 | 120 | 0.20 |
| | IKB-119 | 1000 | 0.030 | 0.030 | 120 | 120 | 2.00 |
| | ASP-120 | 100 | 0.030 | 0.030 | 120 | 120 | 0.20 |
| | ASP-121 | 100 | 0.030 | 0.030 | 120 | 120 | 0.20 |
| | ASP-122 | 100 | 0.015 | 0.015 | 60 | 60 | 0.10 |
| | BLM-123 | 1000 | 0.030 | 0.030 | 120 | 120 | 2.00 |
| | BCK-124 | 100 | 0.030 | 0.030 | 120 | 120 | 0.20 |
| | BCK-125 | 100 | 0.015 | 0.015 | 60 | 60 | 0.10 |
| | BCK-126 | 100 | 0.030 | 0.030 | 120 | 120 | 0.20 |
| | G6P-127 | 100 | 0.030 | 0.030 | 120 | 120 | 0.20 |
| | G6P-128 | 100 | 0.015 | 0.015 | 60 | 60 | 0.10 |
| | ABC-201 | 100 | 0.030 | 0.030 | 120 | 120 | 0.20 |
| | ABC-202 | 100 | 0.030 | 0.030 | 120 | 120 | 0.20 |
| | DLD-203 | 100 | 0.030 | 0.030 | 120 | 120 | 0.20 |
| | DLD-204 | 1000 | 0.030 | 0.030 | 120 | 120 | 2.00 |
| | NEB-205c | 100 | 0.015 | 0.015 | 60 | 60 | 0.10 |
| | NEB-205 | 100 | 0.090 | 0.090 | 360 | 360 | 0.60 |
| | PCD-206 | 100 | 0.030 | 0.030 | 120 | 120 | 0.20 |
| | CLR-207 | 100 | 0.030 | 0.030 | 120 | 120 | 0.20 |
| | TME-208 | 100 | 0.030 | 0.030 | 120 | 120 | 0.20 |
| | FKT-209 | 1000 | 0.015 | 0.015 | 60 | 60 | 1.00 |
| | BetaE1 | 100 | 0.060 | 0.060 | 240 | 240 | 0.40 |
| | BetaE2 | 100 | 0.060 | 0.060 | 240 | 240 | 0.40 |
| | BetaE3 | 100 | 0.060 | 0.060 | 240 | 240 | 0.40 |
| | BetaIVS2 | 100 | 0.030 | 0.030 | 120 | 120 | 0.20 |

| | | | |
|---|---|---|---|
| subtotal (ul) | 2.7 | 1,170 | — |
| Water (ul) | 2.3 | 9,230 | — |
| Total (ul) | 5 | 20,000 | — |

TABLE 10
GSP-B primer pool

| Reagent | GSP-B primer pool (3×) | | | | |
|---|---|---|---|---|---|
| Storage | Store at −10° C. or colder. | | | | |
| Stability | Stable for 1 year. Avoid freeze/thaw cycles. | | | | |
| | | Stock | | | |
| | GSP-B | Conc. | | | Final |
| | primer | (uM) | ul/rxn | ul/4000 rxn | (uM) |
| Preparation | GDL1-for | 100 | 0.03 | 120 | 0.2 |
| | GDL1-rev | 100 | 0.03 | 120 | 0.2 |
| | GDL2-for | 100 | 0.03 | 120 | 0.2 |
| | GDL2-rev | 100 | 0.03 | 120 | 0.2 |
| | BetaE1-Fb | 100 | 0.01 | 36 | 0.06 |
| | BetaE1-Rb | 100 | 0.01 | 36 | 0.06 |
| | BetaE2-Fb | 100 | 0.01 | 36 | 0.06 |
| | BetaE2-Rb | 100 | 0.01 | 36 | 0.06 |
| | BetaE3-Fb | 100 | 0.01 | 36 | 0.06 |
| | BetaE3-Rb | 100 | 0.01 | 36 | 0.06 |

| | | |
|---|---|---|
| Subtotal (ul) | 0.17 | 696 |
| Water (ul) | 4.8 | 19,304 |
| Total (ul) | 5.0 | 20,000 |

TABLE 11
GSP-C primer pool

| Reagent | GSP-C primer pool (3×) | | | | |
|---|---|---|---|---|---|
| Storage | Store at −10° C. or colder. | | | | |
| Stability | Stable for 1 year. Avoid freeze/thaw cycles. | | | | |
| | | Stock | | | |
| | GSP-C | conc. | | | Final |
| | primer | (uM) | ul/rxn | ul/4000 rxn | (uM) |
| Preparation | a2/3.7-F | 100 | 0.06 | 240 | 0.4 |
| | 3.7/20.5-R | 100 | 0.03 | 120 | 0.2 |
| | 4.2-F | 100 | 0.03 | 120 | 0.2 |
| | 4.2-R | 100 | 0.03 | 120 | 0.2 |
| | SEA-F | 100 | 0.09 | 360 | 0.6 |
| | SEA-R | 100 | 0.09 | 360 | 0.6 |
| | THAI-F | 100 | 0.06 | 240 | 0.4 |
| | THAI-R | 100 | 0.06 | 240 | 0.4 |
| | 20.5-F2 | 100 | 0.02 | 60 | 0.1 |
| | MED-F | 100 | 0.02 | 60 | 0.1 |
| | MED-R | 100 | 0.02 | 60 | 0.1 |
| | FIL-F2 | 100 | 0.09 | 360 | 0.6 |
| | FIL-R2 | 100 | 0.09 | 360 | 0.6 |
| | a2-R | 100 | 0.03 | 120 | 0.2 |
| | LIS1-F | 100 | 0.15 | 600 | 1.0 |
| | LIS1-R | 100 | 0.15 | 600 | 1.0 |

| | | |
|---|---|---|
| subtotal | 1.01 | 4020 |
| Water | 4.0 | 15980 |
| Total | 5.0 | 0 |

TABLE 12
BCP-B primer pool

| Reagent | BCP-B primer pool (6×) | | | | |
|---|---|---|---|---|---|
| Storage | Store at −10° C. or colder. | | | | |
| Stability | Stable for 1 year. Avoid freeze/thaw cycles. | | | | |
| | | Stock | | | |
| | BCP-B | conc. | | | Final conc. |
| | primer | (uM) | ul/rxn | ul/4000 rxn | (uM) |
| Preparation | GBA112-F | 100 | 0.004 | 15 | 0.025 |
| | GBA112-R | 100 | 0.004 | 15 | 0.025 |
| | GBA113-F | 100 | 0.004 | 15 | 0.025 |
| | GBA113-R | 100 | 0.004 | 15 | 0.025 |
| | GBA114-F | 100 | 0.008 | 30 | 0.05 |
| | GBA114-R | 100 | 0.008 | 30 | 0.05 |
| | GBA115-F | 100 | 0.008 | 30 | 0.05 |
| | GBA115-R | 100 | 0.008 | 30 | 0.05 |

| | | | |
|---|---|---|---|
| subtotal | 0.045 | 180 | — |
| Water | 2.455 | 9820 | — |
| Total | 2.500 | 10000 | — |

TABLE 13
BCP-C primer pool

| Reagent | BCP-C primer pool (6×) | | | | |
|---|---|---|---|---|---|
| Storage | Store at −10° C. or colder. | | | | |
| Stability | Stable for 1 year. Avoid freeze/thaw cycles. | | | | |
| | | Stock | | | Final |
| | BCP-C | conc. | | ul/4000 | conc. |
| | primer | (uM) | ul/rxn | rxn | (uM) |
| Preparation | 3.7nest-F | 100 | 0.004 | 15 | 0.025 |
| | 3.7/20.5nest-R | 100 | 0.004 | 15 | 0.025 |
| | 4.2nest-F | 100 | 0.008 | 30 | 0.05 |
| | 4.2nest-R | 100 | 0.008 | 30 | 0.05 |
| | SEAnest-F | 100 | 0.008 | 30 | 0.05 |
| | SEAnest-R2 | 100 | 0.008 | 30 | 0.05 |
| | THAInest-F | 100 | 0.004 | 15 | 0.025 |
| | THAInest-R | 100 | 0.004 | 15 | 0.025 |
| | 20.5nest-F | 100 | 0.002 | 7.5 | 0.0125 |
| | 20.5nest-R | 100 | 0.002 | 7.5 | 0.0125 |
| | MEDnest-F | 100 | 0.002 | 7.5 | 0.0125 |
| | MEDnest-R | 100 | 0.002 | 7.5 | 0.0125 |
| | FILnest-F | 100 | 0.004 | 15 | 0.025 |
| | FILnest-R | 100 | 0.004 | 15 | 0.025 |
| | Alnest-F6 | 100 | 0.002 | 7.5 | 0.0125 |
| | a2nest-R | 100 | 0.002 | 7.5 | 0.0125 |
| | LISnest-F | 100 | 0.008 | 30 | 0.05 |
| | LISnest-R | 100 | 0.008 | 30 | 0.05 |

| | | | |
|---|---|---|---|
| subtotal (ul) | 0.079 | 315 | — |
| Water (ul) | 2.421 | 9685 | — |
| Total (ul) | 2.5 | 10000 | — |

TABLE 14
Controls Used

| | |
|---|---|
| Quality | Supplier & Catalog Number ? |
| Control | |
| Phi X | Supplier: Illumina |
| Control | Catalog#: FC-110-3001 |
| v3 | PhiX must be included in every run to serve as a |
| | control for sequencing and library preparation. PhiX |
| | also serves to increase the diversity of the library. |
| Positive | A genomic specimen positive for one of the 10 large |
| Control | deletions (HEXA 7.6 kb del, MCOLN1 6.4 kb del, |
| | NEB 2.5 kb del, and Alpha-3.7, -4.2, -SEA, -THAI, |
| | -20.5, -MED, and -FIL) will be included in every run |
| | on a rotational basis. In addition, a genomic DNA |
| | specimen positive for one of the target variants in this |
| | panel will also be included on a rotational basis in |
| | each run. |
| Negative | No DNA control must be placed at the end of the run. |
| Control | |
| Positional | A QC blank is placed randomly within each plate to |
| Control | ensure results reflect the correct positioning of the |
| | Extraction/PCR plate for detection. |

### Analytical Measurement Range (AMR)

Negative (Wild type): none of the tested variants are detected. Heterozygous: positive for a variant with a frequency between ≥20% and <96%. Compound heterozygous: positive for two variations, one on each allele, within a gene with frequencies between ≥20% and <96%. Homozygous: positive for the same variant on both alleles with a frequency ≥96%.

TABLE 15
Disorders detected by the instant methods and their carrier
frequencies.

| Disorders/ | | Carrier | |
|---|---|---|---|
| Gene | Ethnicity | Frequency | |
| Bloom Syndrome/BLM | Ashkenazi Jewish | 1 in | 100 |
| | General population | <1 in | 500 |
| Canavan Disease/ASPA | Ashkenazi Jewish | 1 in | 41 |
| | General population | <1 in | 158 |
| Familial Dysautonomia | Ashkenazi Jewish | 1 in | 52 |
| (FD)/IKBKAP | General population | 1 in | 166 |
| Fanconi Anemia Type | Ashkenazi Jewish | 1 in | 89 |
| C/FANCC | General population | <1 in | 790 |
| Gaucher Disease/GBA | Ashkenazi Jewish | 1 in | 18 |
| | General population | 1 in | 112 |
| Glycogen Storage disease | Ashkenazi Jewish | 1 in | 71 |
| Type IA (GSDIA)/G6PC | General population | 1 in | 177 |
| Maple Syrup Urine Disease | Ashkenazi Jewish | 1 in | 81 |
| (MSUD)/BCKDHB | General population | 1 in | 240 |
| Mucolipidosis IV | Ashkenazi Jewish | 1 in | 127 |
| (ML4)/MCOLN1 | General population | <1 in | 500 |
| Niemann-Pick Disease | Ashkenazi Jewish | 1 in | 90 |
| Type A/SMPD1 | General population | <1 in | 500 |
| Tay-Sachs Disease | Ashkenazi Jewish | 1 in | 31 |
| (enzyme and DNA)/HEXA | General population | 1 in | 300 |
| Dihydrolipoamide | Ashkenazi Jewish | 1 in | 107 |
| Dehydrogenase | | | |
| Deficiency/DLD | General population | <1 in | 500 |
| Familial Hyperinsulinism | Ashkenazi Jewish | 1 in | 52 |
| (FHI)/ABCC8 | General population | 1 in | 166 |
| Joubert Syndrome/ | Ashkenazi Jewish | 1 in | 95 |
| TMEM216 | General population | <1 in | 500 |
| Nemaline Myopathy | Ashkenazi Jewish | 1 in | 108 |
| (NM)/NEB | General population | <1 in | 500 |
| Usher Syndrome Type IF/ | Ashkenazi Jewish | 1 in | 147 |
| PCDH15 | General population | 1 in | 395 |
| Usher Syndrome Type III/ | Ashkenazi Jewish | 1 in | 120 |
| CLRN1 | General population | <1 in | 500 |
| Walker-Warburg | Ashkenazi Jewish | 1 in | 150 |
| Syndrome/FKTN | General population | <1 in | 500 |

| | | |
|---|---|---|
| Alpha thalassemina/HBA | General population | varied |
| Beta thalassemina/HBB | General population | varied |

### Example 5: Results

The clinical sensitivity and specificity of the disclosed assay was measured to be 100% and 100%, respectively. The assay also demonstrated 100% inter- and intra-assay precision during validation runs.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1.-33. (canceled)

34. A method for detecting one or more gene variants for a genetic disorder in a subject that is suspected of being a carrier for at least one inherited genetic mutation comprising:
extracting DNA from a biological sample obtained from the subject;
generating a first plurality of amplicons by contacting the biological sample with a first plurality of primer pairs, wherein at least one amplicon corresponds to each of a plurality of genes, said plurality of genes comprising HEXA, SMPD1, MCOLN1, GBA, FANCC, IKBKAP, ASPA, BLM, BCKDHB, G6PC, ABCC8, DLD, NEB, PCDH15, CLRN1, TMEM216, and FKTN;
generating a second plurality of amplicons by contacting the biological sample with a second plurality of primer pairs, wherein at least one amplicon corresponds to each of GBA and HBB, wherein the second plurality of primer pairs comprises SEQ ID NOs: 81 and 82, and SEQ ID NOs: 83 and 84; and
generating a third plurality of amplicons by contacting the biological sample with a third plurality of primer pairs, a hotstart Taq DNA polymerase, and a Taq Extender PCR additive, wherein at least one amplicon corresponds to each of HBA1 and HBA2;
incorporating a barcode sequence on to the ends of the first, second and third plurality of amplicons via a polymerase chain reaction; and
detecting one or more gene variants in at least one of the first, second and third plurality of amplicons using high throughput massive parallel sequencing.

35. The method of claim 34, wherein:
the first plurality of primer pairs comprises at least one gap primer pair designed to amplify target regions only when a deletion is present;
the GBA primers of the second plurality of primer pairs specifically amplify the GBA gene and not amplify any GBA pseudogenes; and/or
the third plurality of primer pairs comprises at least one gap primer pair designed to amplify target regions only when a deletion is present.

36. The method of claim 34, wherein:
the HEXA gene variants comprise R178H (B1 variant), R247W, R249W, G269S, IVS9+1G>A, 1278_TATC, IVS12+1G>C, or 7.6-kb Del, Ex1;
the SMPD1 gene variants comprise L302P, fsP330, R496L (R498L), or deltaR608;
the MCOLN1 gene variants comprise IVS3−2A>G, or 6.4 kb_del;
the GBA gene variants comprise IVS2+1G>A, 84G>GG, N370S, del_55 bp, V394L, D409H, L444P, or R496H;
the PANCC gene variants comprise IVS4+4A>T, or 322delG;
the IKBKAP gene variants comprise R696P, or IVS20+6T>C;
the ASPA gene variants comprise IVS2−2A>G, Y231X, E285A, or A305E;
the BLM gene variants comprise 2281del6/ins7;
the BCKDHB gene variants comprise R183P, G278S, or E372X;
the G6PC gene variants comprise R83C or Q347X;
the ABCC8 gene variants comprise IVS32−9G>A or F1387del;
the DLD gene variants comprise Y35* or G229C;
the NEB gene variants comprise R2478_D2512del35;
the PCDH15 gene variants comprise R245;
the CLRN1 gene variants comprise N48K;
the TMEM216 gene variants comprise R73L; and
the FKTN gene variants comprise F390fs.

37. The method of claim 34, the HBB gene variants comprise c.*111A>G, c.*110T>C, c.*96T>C, Hb D-Los Angeles (c.364G>C), Hb O-Arab (c.364G>A), c.321_322insG, c.316−2A>C, c.316−2A>G, c.316−3C>A, c.316−106C>G, c.316−125A>G, c.316−146T>G, c.316−197C>T, c.315+1G>A, c.287_288insA, c.251delG, c.230delC, c.216_217insA, c.203_204delTG, c.143_144insA, c.146_147insATCT, c.135delC, c.130G>T, c.126_129delCTTT, c.124_127delTTCT, c.118C>T, c.114G>A, c.112delT, c.93−1G>C, c.93−1G>A, c.93−21G>A, c.92+6T>C, c.92+5G>A, c.92+5G>C, c.92+5G>T, c.92+2T>A, c.92+2T>C, c.92+1G>A, c.92+1G>T, Hb Monroe (c.92G>C), c.92G>A, c.84_85insC, c.79G>T, HB E (c.79G>A), c.75T>A, c.59A>G, c.52A>T (LYS17*), c.51delC, c.48G>A, c.47G>A (Trp15), c.46delT, c.36delT, c.33C>A, c.27_28insG, c.25_26delAA, c.20delA, HB S (c.20A>T), HB C (c.19G>A), c.17_18delCT, c.2T>C, c.2T>G, c.1A>G, c.−78A>C, c.−78A>G, c.−79A>G, c.−80T>A, c.−81A>G, c.−136C>G, c.−137C>A, c.−137C>G, c.−137C>T, c.−138C>T, c.−138C>A, c.−140C>T, or c.−151C>T.

38. The method of claim 34, wherein the HBA1 and HBA2 genes variants comprise large deletions selected from variant 3.7, variant 4.2, variant SEA, variant THAI, variant 20.5, variant MED or variant FIL; or a single nucleotide variant in the HBA2 gene HBA2 c.427T>C.

39. The method of claim 34, wherein the first plurality of primer pairs comprises at least two primer pairs having at least 85% identity to at least two primer pairs selected from Table 4.

40. The method of claim 34, wherein the second plurality of primer pairs comprises at least two primer pairs having at least 85% identity to at least two primer pairs selected from SEQ ID NOs: 73 and 74, SEQ ID Nos: 75 and 76, SEQ ID NOs: 77 and 78, SEQ ID Nos: 79 and 80, SEQ ID NOs: 85 and 86, SEQ ID NOs: 87 and 88, or SEQ ID NOs: 89 and 90.

41. The method of claim 34, wherein the third plurality of primer pairs comprises at least two primer pairs having at least 85% identity to at least two primer pairs selected from Table 6.

42. The method of claim 34, wherein the high throughput massive parallel sequencing comprises sequencing by synthesis or sequencing by ligation.

43. The method of claim 34, wherein the subject is of Ashkenazi Jewish descent or has a family history of beta thalassemia, or alpha thalassemia.

44. The method of claim 34, wherein the biological sample is whole blood, serum, plasma, amniotic fluid, or chorionic villi.

45. The method of claim 34, wherein the disorder is an autosomal or X-linked recessive disorder.

46. The method of claim 34, wherein the subject is suspected of being a carrier for at least one disease selected from Tay-Sachs disease, Niemann Pick disease, Mucolipidosis Type IV, Gaucher disease, Fanconi Anemia, Familial Dysautonomia, Canavan disease, Bloom syndorme, Maple serum urinary disease, Glycogen storage disease I, Familial Hyperinsulinism, Dihydrolipoamide Dehydrogenase Deficiency (DLD), Lipoamide Dehydrogenase Deficiency (E3), Nemaline Myopathy, Usher Syndrome Type IF, Usher Syndrome, Type IIIA, Joubert Syndrome 2, Walker-Warburg Syndrome, beta thalassemia, or alpha thalassemia.

47. A kit comprising:
(i) a first plurality of primer pairs directed to amplifying regions of each of a plurality of genes comprising gene variants, said plurality of genes comprising HEXA, SMPD1, MCOLN1, GBA, FANCC, IKBKAP, ASPA, BLM, BCKDHB, G6PC, ABCC8, DLD, NEB, PCDH15, CLRN1, TMEM216, and FKTN;
(ii) a second plurality of primer pairs directed to amplifying regions of GBA and HBB genes, wherein the second plurality of primer pairs comprises SEQ ID NOs: 81 and 82, and SEQ ID NOs: 83 and 84; and
(iii) a third plurality of primer pairs directed to amplifying regions of alpha thalassemia gene variants in each of HBA1 and HBA2 genes,
and instructions for use.

48. The kit of claim 47, wherein:
the first plurality of primer pairs comprises at least one gap primer pair designed to amplify target regions only when a deletion is present;
the GBA primers of the second plurality of primer pairs specifically amplify the GBA gene and not amplify any GBA pseudogenes; and/or
the third plurality of primer pairs comprises at least one gap primer pair designed to amplify target regions only when a deletion is present.

49. The kit of claim 47, wherein:
the HEXA gene variants comprise R178H (B1 variant), R247W, R249W, G269S, IVS9+1G>A, 1278_TATC, IVS12+1G>C, or 7.6-kb Del, Ex1;
the SMPD1 gene variants comprise L302P, fsP330, R496L (R498L), or deltaR608;
the MCOLN1 gene variants comprise IVS3−2A>G, or 6.4 kb_del;
the GBA gene variants comprise IVS2+1G>A, 84G>GG, N370S, del_55 bp, V394L, D409H, L444P, or R496H;
the PANCC gene variants comprise IVS4+4A>T, or 322delG;
the IKBKAP gene variants comprise R696P, or IVS20+6T>C;
the ASPA gene variants comprise IVS2−2A>G, Y231X, E285A, or A305E;
the BLM gene variants comprise 2281del6/ins7;
the BCKDHB gene variants comprise R183P, G278S, or E372X;
the G6PC gene variants comprise R83C or Q347X;
the ABCC8 gene variants comprise IVS32−9G>A or F1387del;
the DLD gene variants comprise Y35* or G229C;
the NEB gene variants comprise R2478_D2512del35;
the PCDH15 gene variants comprise R245;
the CLRN1 gene variants comprise N48K;
the TMEM216 gene variants comprise R73L; and
the FKTN gene variants comprise F390fs.

50. The kit of claim 47, the HBB gene variants comprise c.*111A>G, c.*110T>C, c.*96T>C, Hb D-Los Angeles (c.364G>C), Hb O-Arab (c.364G>A), c.321_322insG, c.316−2A>C, c.316−2A>G, c.316−3C>A, c.316−106C>G, c.316−125A>G, c.316−146T>G, c.316−197C>T, c.315+1G>A, c.287_288insA, c.251delG, c.230delC, c.216_217insA, c.203_204delTG, c.143 144insA, c.146_147insATCT, c.135delC, c.130G>T, c.126_129delCTTT, c.124_127delTTCT, c.118C>T, c.114G>A, c.112delT, c.93−1G>C, c.93−1G>A, c.93−21G>A, c.92+6T>C, c.92+5G>A, c.92+5G>C, c.92+5G>T, c.92+2T>A, c.92+2T>C, c.92+1G>A, c.92+1G>T, Hb Monroe (c.92G>C), c.92G>A, c.84_85insC, c.79G>T, HB E (c.79G>A), c.75T>A, c.59A>G, c.52A>T (LYS17*), c.51delC, c.48G>A, c.47G>A (Trp15), c.46delT, c.36delT, c.33C>A, c.27_28insG, c.25_26delAA, c.20delA, HB S (c.20A>T), HB C (c.19G>A), c.17_18delCT, c.2T>C, c.2T>G, c.1A>G, c.−78A>C, c.−78A>G, c.−79A>G, c.−80T>A, c.−81A>G, c.−136C>G, c.−137C>A, c.−137C>G, c.−137C>T, c.−138C>T, c.−138C>A, c.−140C>T, or c.−151C>T; and
the HBA1 and HBA2 gene variants comprise large deletions selected from variant 3.7, variant 4.2, variant SEA, variant THAI, variant 20.5, variant MED or variant FIL; or a single nucleotide variant in the HBA2 gene HBA2 c.427T>C.

51. The kit of claim 47, wherein the first plurality of primer pairs comprises at least two primer pairs having at least 85% identity to at least two primer pairs selected from Table 4.

52. The kit of claim 47, wherein the second plurality of primer pairs comprises at least two primer pairs having at least 85% identity to at least two primer pairs selected from SEQ ID NOs: 73 and 74, SEQ ID Nos: 75 and 76, SEQ ID NOs: 77 and 78, SEQ ID Nos: 79 and 80, SEQ ID NOs: 85 and 86, SEQ ID NOs: 87 and 88, or SEQ ID NOs: 89 and 90.

53. The kit of claim 47, wherein the third plurality of primer pairs comprises at least two primer pairs having at least 85% identity to at least two primer pairs selected from Table 6.
